(19) United States
(12) Patent Application Publication
(10) Pub. No.: US 2022/0369919 A1
(43) Pub. Date: Nov. 24, 2022

(54) ENDOSCOPIC DEVICES, ACCESS SHEATHS, AND ASSOCIATED METHODS

(71) Applicant: BOSTON SCIENTIFIC SCIMED, INC., Maple Grove, MN (US)

(72) Inventors: MICHAEL S. H. CHU, Brookline, MA (US); ERIC NOBLE LENT, Roberts, WI (US); NIRAJ PRASAD RAUNIYAR, Plymouth, MN (US); JOHN SUBASIC, Hudson, MA (US); ELIZABETH A. STOKLEY, Raleigh, NC (US); ANANT VAIDYANATHAN SUBRAMANIAM, Brighton, MA (US); WILLIAM STANHOPE, Lunenburg, MA (US); KIMBERLY DeGRAAF, Holden, MA (US); SHARMAD S. JOSHI, Auburndale, MA (US); ADAM PERRY NODIFF, Southborough, MA (US); RONGWEI JASON XUAN, Fremont, CA (US); LANE JACOBS, Fruit Heights, UT (US)

(73) Assignee: BOSTON SCIENTIFIC SCIMED, INC., Maple Grove, MN (US)

(21) Appl. No.: 17/747,667

(22) Filed: May 18, 2022

Related U.S. Application Data
(60) Provisional application No. 63/190,546, filed on May 19, 2021.

(51) Int. Cl. A61B 1/307; A61B 1/005; A61B 1/018; A61B 1/00

(52) U.S. Cl. CPC A61B 1/307; A61B 1/0052; A61B 1/018; A61B 1/00103; A61B 1/00105

(57) ABSTRACT

Systems and methods for treating a patient may include accessing an anatomical structure with a single use visualization device, using the imaging capabilities of the single use visualization device within the anatomical structure, and in some cases, advancing the single use visualization device beyond the anatomical structure to and toward another anatomical structure within the patient. Disposable elements may be used with or added to the single use visualization device to accomplish tasks normally performed with reusable devices.

### CROSS REFERENCE TO RELATED APPLICATIONS

This application claims the benefit of and priority to U.S. Provisional Patent Application Ser. No. 63/190,546 filed on May 19, 2021, the disclosure of which is incorporated herein by reference.

### TECHNICAL FIELD

The present disclosure pertains to medical devices and systems and methods for manufacturing and using medical devices and systems. More particularly, the present disclosure pertains to endoscopic devices, such as ureteroscopes and methods of manufacturing and using such endoscopic devices.

### BACKGROUND

Endoscopic devices are used for various diagnostic and surgical procedures. For example, flexible ureteroscopes are utilized in the examination and treatment of kidneys and may generally include features which improve treatment site accessibility and patient comfort. Flexible endoscopic devices may be provided with a flexible tip section that is controlled by a physician via manipulation of various components attached to the scope's handle. Such manipulation enables the physician to maneuver the tip of the scope to different locations within the body (e.g., different locations within the kidney). Additionally, endoscopes are typically used in conjunction with other medical devices during a medical procedure. For example, urologists may use a flexible ureteroscope in combination with both a laser fiber and a retrieval device (e.g., retrieval basket) to pulverize kidney stones and/or to remove debris from the body. Accordingly, these procedures may require not only manipulating various features of the endoscope to control the tip of the scope, but also to introduce and manipulate ancillary devices used in conjunction with the endoscope. In some cases, physicians may use a reusable cystoscope to access the bladder before inserting the flexible ureteroscope through the reusable cystoscope to access the kidney. Such procedures may duplicate functions (e.g., scope functions) while increasing set-up times and the cost to perform the procedure. Reducing device costs, maximizing patient safety, and reducing procedure time and complication each offer clear benefits to the practitioner.

Of the known medical devices and methods, each has certain advantages and disadvantages. There is an ongoing need to provide alternative medical devices as well as alternative methods for manufacturing and using medical devices.

### BRIEF SUMMARY

This disclosure provides design, material, manufacturing method, and use alternatives for medical devices, including endoscopic devices, endoscopic systems, and endoscopic accessories.

A first example is a medical system. The medical system includes an access sheath, a bridge, and a flexible ureteroscope. The access sheath has a lumen extending from a proximal end to a distal opening. The bridge is secured to the proximal end of the access sheath. The bridge includes a lumen extending therethrough in fluid communication with the lumen of the access sheath. The flexible ureteroscope includes a handle and an elongate shaft extending distally from the handle. The elongate shaft has a length and includes a deflectable distal tip. The elongate shaft is configured to be slidably disposed within the lumen of the access sheath and the lumen of the bridge. The elongate shaft of the flexible ureteroscope includes at least one crosshair marking disposed on an outer surface of the elongate shaft and configured to position the deflectable distal tip of the elongate shaft at the distal opening of the access sheath. When the deflectable distal tip of the elongate shaft is positioned at the distal opening of the access sheath, at least 20% of the length of the elongate shaft extends proximal of the bridge.

Alternatively or additionally to any of the examples above, in another example, the deflectable distal tip of the elongate shaft is positioned at the distal opening of the access sheath, at least 30% of the length of the elongate shaft extends proximal of the bridge.

Alternatively or additionally to any of the examples above, in another example, the system includes an access sheath configured to be slidably disposed within the lumen of the access sheath simultaneously with the elongate shaft of the flexible ureteroscope.

Another example is a medical system for treating a urinary tract of a patient. The system includes a flexible ureteroscope and an elongate adapter element. The flexible ureteroscope includes an elongate shaft having a deflectable distal tip, a working lumen extending therethrough, and optics disposed in the deflectable distal tip and in electronic communication with a handle of the flexible ureteroscope. The elongate adapter element is configured to removably attach to the elongate shaft. The elongate adapter element includes a working channel extending from a proximal end of the elongate adapter element to a distal end of the elongate adapter element and an open slot extending along a majority of a length of the elongate adapter element. The elongate adapter element is configured to apply a radially inward force on an outer surface of the elongate shaft when at least a portion of the elongate shaft is disposed within the open slot. The working channel is circumferentially enclosed from the proximal end of the elongate adapter element to the distal end of the elongate adapter element and has a first cross-sectional area. The open slot has a second cross-sectional area greater than the first cross-sectional area.

Alternatively or additionally to any of the examples above, in another example, when at least a portion of the elongate shaft is disposed within the open slot, the elongate adapter element extends circumferentially around at least 60% of a circumference of the elongate shaft.

Alternatively or additionally to any of the examples above, in another example, when at least a portion of the elongate shaft is disposed within the open slot, the elongate adapter element extends circumferentially around at least 75% of a circumference of the elongate shaft.

Alternatively or additionally to any of the examples above, in another example, the elongate adapter element is configured to be positioned proximal of the deflectable distal tip when at least a portion of the elongate shaft is disposed within the open slot.

Alternatively or additionally to any of the examples above, in another example, the elongate adapter element includes a support member configured to secure excess length of the elongate shaft relative to the elongate adapter element.

Alternatively or additionally to any of the examples above, in another example, the support member forms the excess length of the elongate shaft into a loop.

Alternatively or additionally to any of the examples above, in another example, the elongate adapter element includes an attachment portion configured to removably attach to the handle of the flexible ureteroscope.

Alternatively or additionally to any of the examples above, in another example, the elongate adapter element includes a laterally facing cutout in communication with the open slot and disposed between the proximal end of the elongate adapter element and a longitudinal midpoint of the elongate adapter element.

Alternatively or additionally to any of the examples above, in another example, when at least a portion of the elongate shaft is disposed within the open slot, a portion of the elongate shaft extends through the laterally facing cutout and is positioned outside of the elongate adapter element.

Another example is a modular medical device. The modular medical device includes a handle portion and a disposable shaft portion. The disposable shaft portion is configured to releasably connect to the handle portion. The handle portion includes electronic components configured to communicate with the disposable shaft portion when the disposable shaft portion is connected to the handle portion. The handle portion includes a proximal working channel extending through the handle portion. The disposable shaft portion includes a distal working channel configured to be in fluid communication with the proximal working channel when the disposable shaft portion is connected to the handle portion. The disposable shaft portion includes illumination and imaging components disposed at a distal end of the disposable shaft portion. The illumination and imaging components are in electronic communication with the electronic components when the disposable shaft portion is connected to the handle portion. The illumination and imaging components are configured to illuminate and image an area proximate the distal end of the disposable shaft portion.

Alternatively or additionally to any of the examples above, in another example, the disposable shaft portion includes a printed circuit board disposed at the proximal end thereof, wherein the printed circuit board is configured to control the illumination and imaging components.

Alternatively or additionally to any of the examples above, in another example, the handle portion includes a reusable handle portion and a disposable handle portion. The proximal working channel is disposed within the disposable handle portion and the electronic components are disposed within the reusable handle portion. When the disposable shaft portion is connected to the handle portion, the proximal end of the disposable shaft portion engages both the disposable handle portion and the reusable handle portion.

Alternatively or additionally to any of the examples above, in another example, the illumination and imaging components are disposed on an imaging assembly removably disposed within an elongate sleeve fixedly disposed within the disposable shaft portion.

Another example is a method of treating a kidney of a patient. The method includes advancing an access sheath through a urethra into a bladder of the patient and disposing a flexible ureteroscope within a lumen of the access sheath. The method further includes visually locating a ureteric orifice within the bladder using the flexible ureteroscope while the flexible ureteroscope is disposed within the lumen of the access sheath. The method further includes inserting a guidewire into a working lumen the flexible ureteroscope and advancing the guidewire from the working lumen into the ureteric orifice while the flexible ureteroscope is disposed within the lumen of the access sheath. The method further includes advancing the flexible ureteroscope through the ureteric orifice and into the kidney while the flexible ureteroscope is disposed within the lumen of the access sheath and performing a procedure within the kidney using the flexible ureteroscope while the flexible ureteroscope is disposed within the lumen of the access sheath.

Alternatively or additionally to any of the examples above, in another example, the method of treating a kidney of a patient includes removing the flexible ureteroscope from the lumen of the access sheath while maintaining a distal end of the guidewire within the kidney and advancing a deflectable distal tip of the flexible ureteroscope through the lumen of the access sheath alongside the guidewire and into the bladder.

Alternatively or additionally to any of the examples above, in another example, the method of treating a kidney of a patient includes advancing a stent over the guidewire while the flexible ureteroscope is disposed within the lumen of the access sheath and positioning a distal end of the stent within the kidney while the flexible ureteroscope is disposed within the lumen of the access sheath.

Alternatively or additionally to any of the examples above, in another example, the method of treating a kidney of a patient includes visually confirming placement of the stent using the flexible ureteroscope while the flexible ureteroscope is disposed within the lumen of the access sheath.

Alternatively or additionally to any of the examples above, in another example, the method of treating a kidney of a patient that includes disposing a flexible ureteroscope within a lumen of the access sheath includes aligning at least one crosshair marking on an elongate shaft of the flexible ureteroscope with a corresponding crosshair marking on the access sheath and locking an orientation of the flexible ureteroscope relative to the access sheath.

Alternatively or additionally to any of the examples above, in another example, the method of treating a kidney of a patient includes disposing an access sheath over the guidewire and advancing the access sheath into the ureteric orifice while the flexible ureteroscope is disposed within the lumen of the access sheath.

Alternatively or additionally to any of the examples above, in another example, the method of treating a kidney of a patient that includes disposing an access sheath over the guidewire includes disposing the access sheath over an elongate shaft of the flexible ureteroscope, wherein advancing the access sheath into the ureteric orifice while the flexible ureteroscope is disposed within the lumen of the access sheath includes advancing the access sheath over the elongate shaft of the flexible ureteroscope into the ureteric orifice after advancing the flexible ureteroscope through the ureteric orifice and into the kidney while the flexible ureteroscope is disposed within the lumen of the access sheath.

The above summary of some embodiments, aspects, and/or examples is not intended to describe each embodiment or every implementation of the present disclosure.

The figures and the detailed description which follows more particularly exemplify these embodiments.

### BRIEF DESCRIPTION OF THE DRAWINGS

The disclosure may be more completely understood in consideration of the following detailed description in connection with the accompanying drawings. It is noted that all figures are not necessarily drawn to scale and/or selected elements may change scale within the figure in order to facilitate a clear view of the elements and/or a clear understanding of the elements. Changes in scale will be apparent to the skilled artisan and need not be expressly identified.

FIG. 1 illustrates an example multi-scope medical system;

FIG. 2 illustrates an example access sheath and dilator;

FIG. 3 illustrates the example access sheath shown in FIG. 2 and a pinch valve bridge;

FIG. 4 is a cross-sectional view of the pinch valve bridge shown in FIG. 3;

FIG. 5 illustrates the example access sheath shown in FIG. 2 and an example Y-bridge;

FIG. 6 illustrates the example access sheath shown in FIG. 2 and an example Y-bridge;

FIGS. 7-13 illustrate aspects of an example medical system and methods of using the medical system;

FIGS. 14-15 illustrate aspects of an example medical system and methods of using the medical system;

FIG. 16 is a side view of an elongate adapter element attached to the elongate shaft of a flexible ureteroscope;

FIG. 17 is a perspective view of the elongate adapter element of FIG. 16;

FIG. 18 is a perspective view of a distal portion of the elongate adapter element of FIG. 16 attached to a flexible ureteroscope;

FIG. 19 illustrates an example support member associated with the elongate adapter element of FIG. 16;

FIG. 20 illustrates an example support member associated with the elongate adapter element of FIG. 16;

FIG. 21 illustrates an example support member associated with the elongate adapter element of FIG. 16;

FIG. 22 illustrates an example attachment of a proximal portion of the elongate adapter element of FIG. 16 to the handle of a flexible ureteroscope;

FIG. 23 illustrates an example attachment of a proximal portion of the elongate adapter element of FIG. 16 to the handle of a flexible ureteroscope;

FIG. 24 illustrates an example attachment of a proximal portion of the elongate adapter element of FIG. 16 to the handle of a flexible ureteroscope;

FIG. 25 illustrates an example attachment of a proximal portion of the elongate adapter element of FIG. 16 to the handle of a flexible ureteroscope;

FIG. 26 illustrates aspects of a medical system and methods of using the medical system;

FIG. 27 illustrates an alternative configuration of an elongate adapter element attached to a flexible ureteroscope;

FIG. 28 illustrates aspects of a modular medical device;

FIGS. 29-31 illustrate example configurations of a disposable shaft portion of the modular medical device of FIG. 28;

FIG. 32 illustrates an alternative configuration of a handle portion of the modular medical device of FIG. 28;

FIGS. 33-34 illustrates example configurations of an imaging assembly associated with the disposable shaft portion of the modular medical device of FIG. 28; and

FIGS. 35-40 illustrate example distal tip configurations of the imaging assembly of FIGS. 33-34.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate but not limit the current disclosure. Those skilled in the art will recognize that the various elements described and/or shown may be arranged in various combinations and configurations without departing from the scope of the disclosure. The detailed description and drawings illustrate example embodiments of the current disclosure. However, in the interest of clarity and ease of understanding, while every feature and/or element may not be shown in each drawing, the feature(s) and/or element(s) may be understood to be present regardless, unless otherwise specified.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed example(s). Each instance of the features may include and/or be encompassed by the singular disclosure(s), unless expressly stated to the contrary. For simplicity and clarity purposes, not all elements of the current disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The term “extent” may be understood to mean a greatest measurement of a stated or identified dimension, unless the extent or dimension in question is preceded by or identified as a “minimum”, which may be understood to mean a smallest measurement of the stated or identified dimension. For example, “outer extent” may be understood to mean an outer dimension, “radial extent” may be understood to mean a radial dimension, “longitudinal extent” may be understood to mean a longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently—such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete structures or elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to implement the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

The following figures illustrate selected components and/or arrangements of medical devices and/or systems and methods of using the same. It should be noted that in any given figure, some features may not be shown, or may be shown schematically, for simplicity. Additional details regarding some of the components of the system may be illustrated in other figures in greater detail. While discussed in the context of accessing and treating the urinary tract, the system may also be used for other interventions and/or percutaneous medical procedures within a patient. In some instances, the system may be used during a nephrolithotomy (PCNL) procedure where a nephroscope is used. Similarly, the devices and methods described herein with respect to percutaneous deployment may be used in other types of surgical procedures, as appropriate. For example, in some examples, the devices may be used in a non-percutaneous procedure. Devices and methods in accordance with the disclosure may also be adapted and configured for other uses within the anatomy.

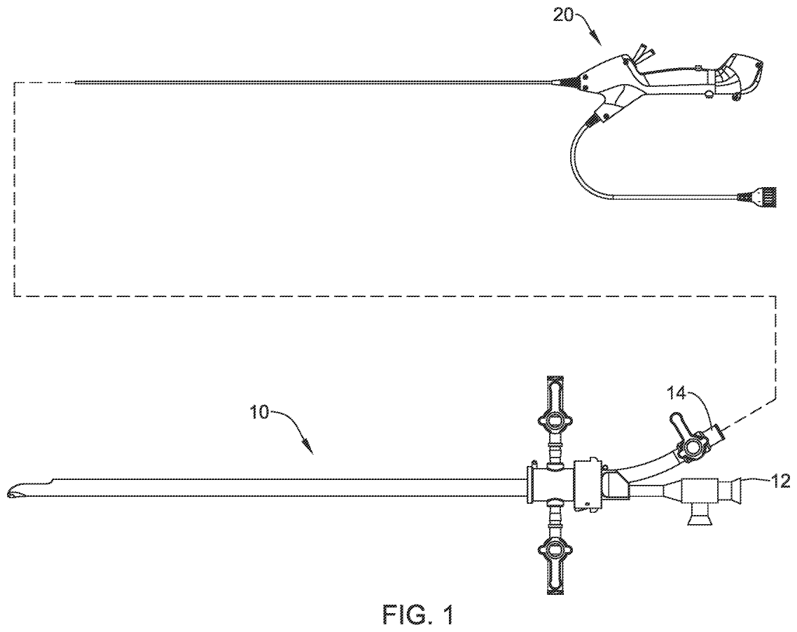

FIG. 1 illustrates a medical system for accessing and treating an anatomical site in a patient's body, such as the urinary tract. For instance, the system may be configured to be used to treat the upper urinary tract, including the kidneys and/or ureters, or the system may be configured to be used to treat the lower urinary tract, including the bladder and/or urethra. As shown, the medical system may include a ureteroscope 20 (which may be a flexible ureteroscope) and a cystoscope 10. The cystoscope 10 includes a camera or imaging system 12 in communication with a working lumen of the cystoscope 10. The cystoscope 10 also includes a side port 14 in communication with the working lumen of the cystoscope 10. The cystoscope 10 is generally rigid (e.g., includes a rigid elongate shaft) and is used by a practitioner to access the bladder of a patient via the urethra. Separate medical devices may then be inserted through the side port 14 to access the urethra, the bladder, the ureters, and/or the kidneys. The ureteroscope 20 may include its own camera or imaging system. As a result of this arrangement, duplication of function and components occurs, which increases the cost of the procedure(s) being performed. In some cases, the imaging components and associated electronics are the most expensive components in the ureteroscope 20 and/or the cystoscope 10.

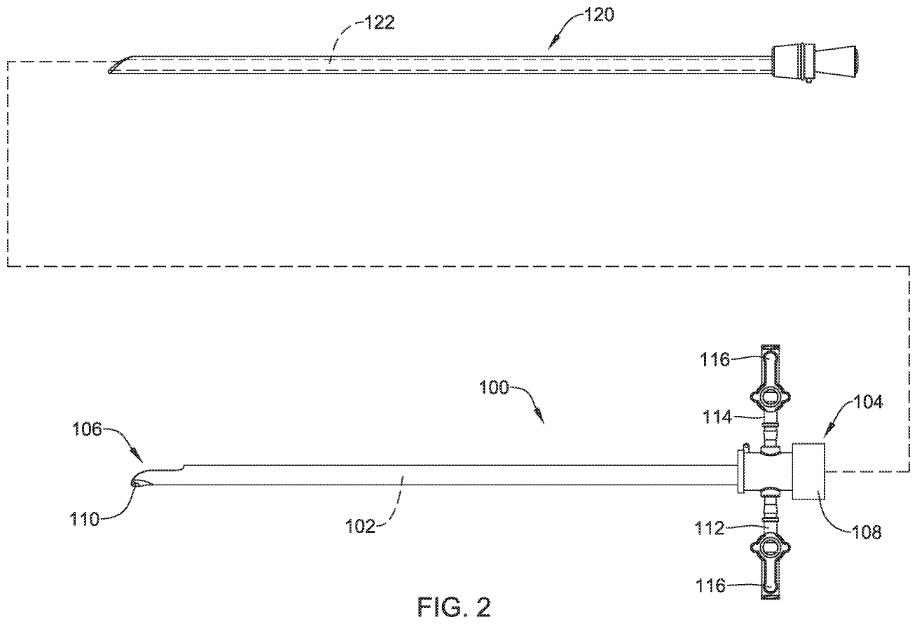

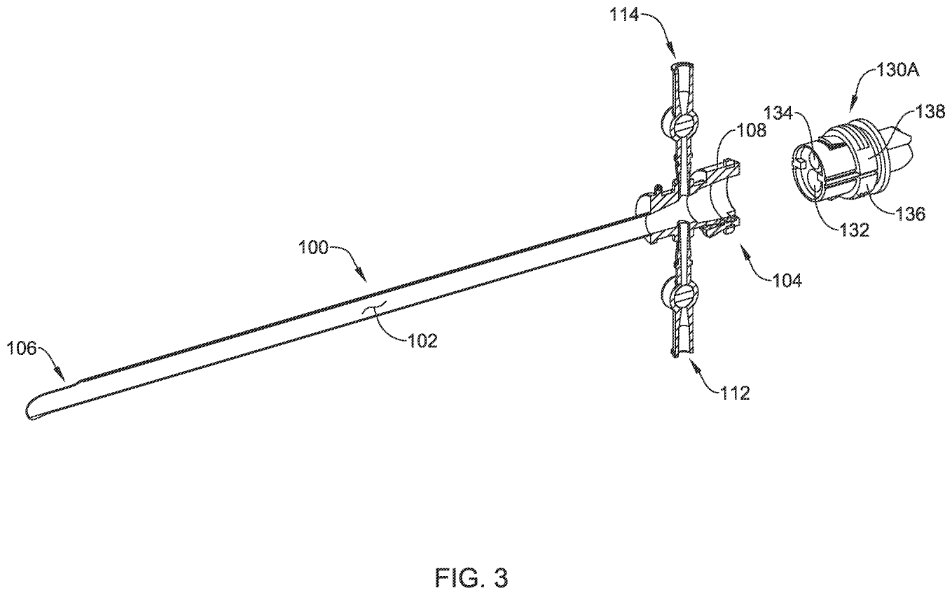

FIG. 2 illustrates an access sheath 100, which in some instances may be referred to as a urosheath, for use in a urology procedure. In some instances, the access sheath 100 may be considered a cystosheath that may be used in place of the cystoscope 10 in a medical system according to the disclosure. In other instances, the access sheath 100 may be considered a nephrosheath that may be used in place of a nephroscope in a medical system according to the disclosure. The access sheath 100 may be a tubular member and may include a lumen 102 (e.g., FIG. 3) extending from a proximal end 104 to a distal opening 106. In some embodiments, the access sheath 100 may have a length of about 9.1 inches (about 23 centimeters). In some embodiments, the access sheath 100 may have a length of about 7.5 inches (about 19 centimeters), about 8 inches (about 20.3 centimeters), about 8.5 inches (about 21.6 centimeters), about 9.5 inches (about 24.1 centimeters), about 10 inches (about 25.4 centimeters), about 10.5 inches (about 26.7 centimeters), etc.

In some embodiments, the medical system may further include an obturator 120. The obturator 120 may be configured to be slidably disposed within the lumen 102 of the access sheath 100 as the access sheath 100 is advanced within a body lumen (e.g., the urethra). In at least some embodiments, the access sheath 100 may be substantially rigid in construction (e.g., includes a rigid elongate shaft). In some embodiments, the access sheath 100 may be formed from a radiopaque material (e.g., a metallic material, a polymeric material with a radiopaque substance mixed in, etc.). In some embodiments, the access sheath 100 may include one or more radiopaque markers secured to and/or embedded within the access sheath 100. In some embodiments, the access sheath 100 may include and/or be formed with a reinforcing braid and/or coil extending longitudinally between the proximal end 104 and a distal end.

The access sheath 100 may include an atraumatic distal tip with a beak 110 and rounded edges to facilitate insertion without the obturator 120. In some embodiments, the distal tip may be formed from or covered with a softer material than the remainder to the access sheath 100. In some embodiments, the access sheath 100 may include a first side port 112 and a second side port 114 in fluid communication with the lumen 102 for providing irrigation and/or aspiration. The first side port 112 and/or the second side port 114 may each optionally include a stopcock 116 or other valve secured thereto to control fluid flow through the first side port 112 and/or the second port side. In some embodiments, the stopcock 116 may be added to the access sheath 100 during a procedure or during preparation for the procedure as needed to reduce cost of the access sheath 100 in procedures that do not require or benefit from fluid flow (e.g., irrigation, aspiration, etc.). Accordingly, in at least some embodiments, the access sheath 100 may be a disposable component of the medical system (e.g., a single-use device).

In some embodiments, the lumen 102 may have a cross-sectional shape that is oval or elliptical, with a major axis extending in a direction between the first side port 112 and the second side port 114 and a minor axis extending perpendicular to the major axis and perpendicular to a central longitudinal axis of the lumen 102 and/or the access sheath 100. Other configurations and/or cross-sectional shapes are also contemplated. The obturator 120 may have a cross-sectional outer shape that is the same as and/or is complimentary to the cross-sectional shape of the lumen 102. In some embodiments, the obturator 120 may include a lumen 122 extending therethrough. The lumen 122 of the obturator 120 may be useful for passing a guidewire, a flexible scope, or other medical device through the obturator 120 to access the bladder and/or the urinary tract of the patient. In some embodiments, the obturator 120 may be formed from a polymeric material. The obturator 120 may be useful for blind insertion into the bladder of female patients. In at least some embodiments, the obturator 120 may be releasably locked to a proximal hub 108 of the access sheath 100 using one or more latches. In some embodiments, a proximal portion of the obturator 120 may have an outer extent and/or shaft profile than a distal portion of the obturator 120. In some embodiments, a distal tip and/or distal end of the obturator 120 may have an outer extent and/or profile that is greater than the outer extent and/or profile of a remainder of the obturator 120 and/or a shaft of the obturator 120.

In at least some embodiments, the medical system may include a bridge 130. Multiple examples of the bridge 130 are described herein. Different examples will be designated using an alpha character attached to the reference number (e.g., bridge 130A, bridge 130B, bridge 130C, etc.). Each may be referred to using the generic reference “bridge 130” in the description and different examples of the bridge 130 may be exchanged and/or interchanged depending upon use and desired features. Furthermore, common and/or similar elements of the various examples of the bridge 130 may be referred to using the same reference number in each example. The bridge 130 may be disposed at and/or secured to the proximal hub 108 of the access sheath 100 (after removal of the obturator 120, if present/used). In some embodiments, the bridge 130 may include a valve (e.g., a Touhy-Borst connector, etc.) configured to secure the obturator 120 relative to the access sheath 100 in a first configuration and configured to permit the obturator 120 to move and/or slide relative to the access sheath 100 in a second configuration. FIG. 3 is an exploded partial cross-sectional view illustrating the access sheath 100 with a pinch valve bridge 130A. As seen in FIG. 3, the lumen 102 may be tapered radially outward in the proximal direction within the proximal hub 108. The proximal hub 108 may be configured to receive and/or secure to the bridge 130. In the example shown in FIG. 3, the pinch valve bridge 130A is configured to releasably attach to the proximal hub 108. In at least some embodiments, the pinch valve bridge 130A may releasably attach to the proximal hub 108 using a twist lock mechanism. In some embodiments, the pinch valve bridge 130A may be fixedly and/or permanently attached to the proximal hub 108 and/or the access sheath 100.

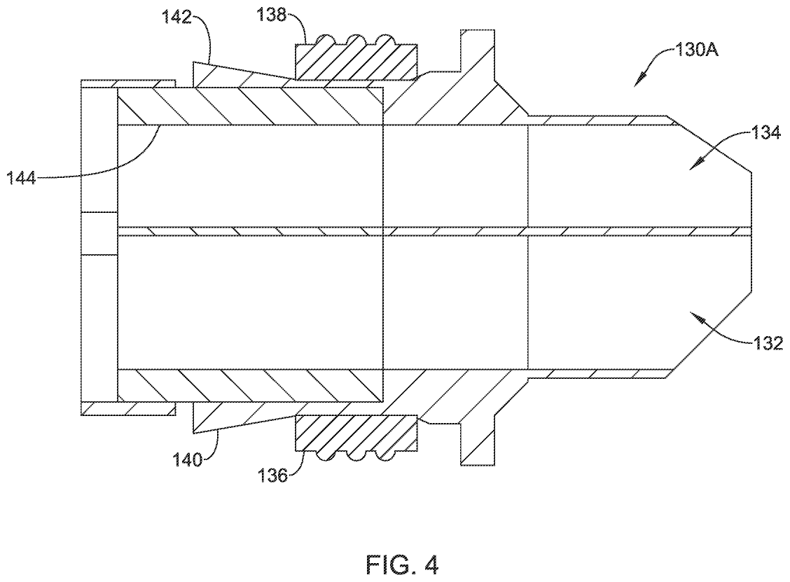

The pinch valve bridge 130A may include a first lumen 132 and a second lumen 134 extending through the pinch valve bridge 130A. The pinch valve bridge 130A may include a first slide 136 and a second slide 138 slidably disposed along an outer surface of the pinch valve bridge 130A. The first slide 136 and the second slide 138 may be configured to move independently of each other. In other examples, the first slide 136 and the second slide 138 may be fixed together, may be formed as a unitary and/or monolithic structure, and/or may move together. The first slide 136 may be associated with the first lumen 132 and the second slide 138 may be associated with the second lumen 134. The pinch valve bridge 130A may include a first deflectable tab 140 associated with the first slide 136 and the first lumen 132, as seen in FIG. 4. The pinch valve bridge 130A may include a second deflectable tab 142 associated with the second slide 138 and the second lumen 134. The pinch valve bridge 130A may include a polymeric sealing element 144 disposed therein. The polymeric sealing element 144 may form at least a portion of the first lumen 132 and/or the second lumen 134. In some embodiments, the first lumen 132 may have an inner extent that is greater than an inner extent of the second lumen 134. In some embodiments, the inner extent of the first lumen 132 may be less than the inner extent of the second lumen 134. In some embodiments, the inner extent of the first lumen 132 may be substantially the same as the inner extent of the second lumen 134.

The first slide 136 may be configured to slide along the outer surface of the pinch valve bridge 130A and over the first deflectable tab 140, thereby deflecting the first deflectable tab 140 radially inward against and/or into contact with the polymeric sealing element 144. Deflecting the first deflectable tab 140 radially inward may deform the polymeric sealing element 144 and/or reduce the inner extent of the first lumen 132 within the polymeric sealing element 144. As will be appreciated, deflecting the first deflectable tab 140 radially inward while a medical device is disposed within the first lumen 132 may effectively deform the polymeric sealing element 144 against the medical device to seal off the first lumen 132. Deflecting the first deflectable tab 140 radially inward while a medical device is disposed within the first lumen 132 may lock an orientation of the medical device relative to the access sheath 100.

Similarly, the second slide 138 may be configured to slide along the outer surface of the pinch valve bridge 130A and over the second deflectable tab 142, thereby deflecting the second deflectable tab 142 radially inward against and/or into contact with the polymeric sealing element 144. Deflecting the second deflectable tab 142 radially inward may deform the polymeric sealing element 144 and/or reduce the inner extent of the second lumen 134 within the polymeric sealing element 144. As will be appreciated, deflecting the second deflectable tab 142 radially inward while a medical device is disposed within the second lumen 134 may effectively deform the polymeric sealing element 144 against the medical device to seal off the second lumen 134. Deflecting the second deflectable tab 142 radially inward while a medical device is disposed within the second lumen 134 may lock an orientation of the medical device relative to the access sheath 100.

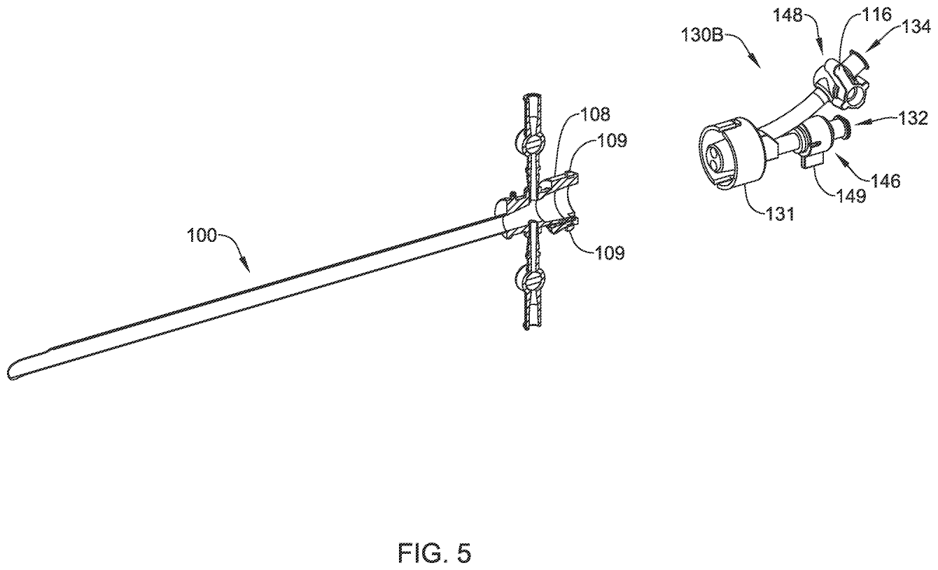

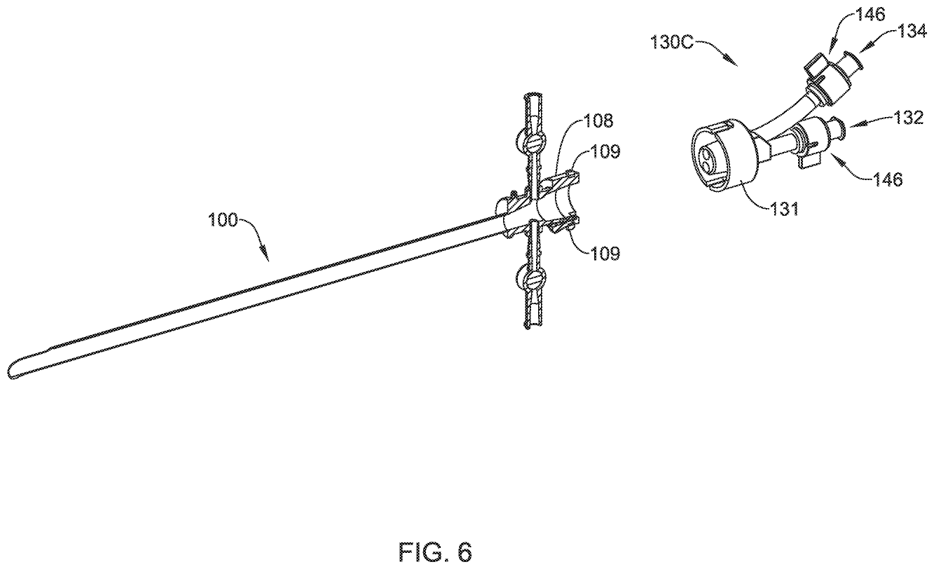

FIG. 5 illustrates the access sheath 100 with a bridge 130B. The bridge 130B may be a Y-bridge. The bridge 130B may include a first lumen 132 and a second lumen 134. In some embodiments, the first lumen 132 may be a straight lumen and the second lumen 134 may be a curved and/or angled lumen. The bridge 130B may include a first fitting 146 associated with the first lumen 132 and a second fitting 148 associated with the second lumen 134. Various types of fittings may be used for the first fitting 146 and the second fitting 148. In the illustrated example, the first fitting 146 may include a cam fitting including a lever 149 configured to displace one or more plungers radially inward against a polymeric sealing member disposed therein when actuated to open and close the first lumen 132, to seal against a medical device disposed within the first lumen 132, and/or to lock an orientation of the medical device relative to the access sheath 100. In the illustrated example of FIG. 5, the second fitting 148 may include a stopcock 116 configured to open and close the second lumen 134, to seal against a medical device disposed within the second lumen 134, and/or to lock an orientation of the medical device relative to the access sheath 100 when actuated. In an alternative example shown in FIG. 6, a bridge 130C may be a Y-bridge including two first fittings 146, one associated with each of the first lumen 132 and the second lumen 134. Other configurations are also contemplated. In some embodiments, the bridge 130B/130C may be configured to releasably attach to the proximal hub 108. In some embodiments, the bridge 130B/130C may releasably attach to the proximal hub 108 using a twist lock mechanism. In some embodiments, the twist lock mechanism may include a plurality of tabs 109 extending radially outward from the proximal hub 108 and a plurality of L-shaped slots formed in a distal collar 131 of the bridge 130B/130C, wherein the plurality of L-shape slots is configured to receive the plurality of tabs 109 therein. Other configurations are also contemplated. In some embodiments, the bridge 130B/130C may be fixedly and/or permanently attached to the proximal hub 108 and/or the access sheath 100.

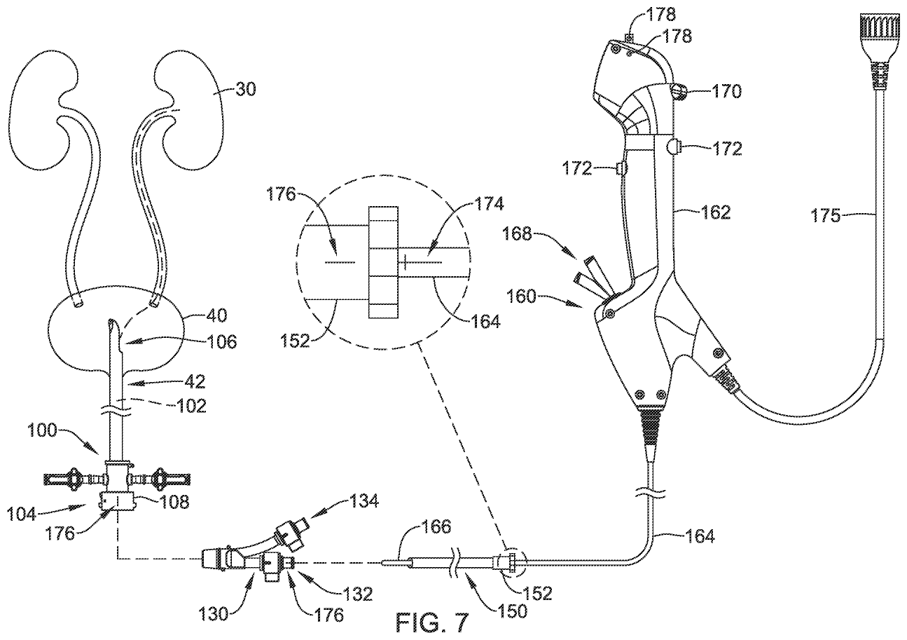

FIG. 7 illustrates aspects of the medical system in use. The medical system may include the access sheath 100 described herein. In some embodiments, a method of using the medical system and/or a method of treating the urinary tract of a patient, such as a kidney 30 of a patient, in a retrograde approach may include advancing the atraumatic distal tip of the access sheath 100 through a urethra 42 into a bladder 40 of the patient. When treating a female patient, the obturator 120 may be disposed within the lumen 102 of the access sheath 100 while advancing the access sheath 100 into the bladder 40. The method may include subsequently removing the obturator 120 from the lumen 102 of the access sheath 100.

In some embodiments, the medical system may include the bridge 130 releasably secured to the proximal end 104 and/or the proximal hub 108 of the access sheath 100. The bridge 130 may include a lumen (e.g., the first lumen 132, the second lumen 134, etc.) extending therethrough in fluid communication with the lumen 102 of the access sheath 100. In some embodiments, the bridge 130 may include multiple lumens (e.g., the first lumen 132, the second lumen 134, etc.) extending therethrough in fluid communication with the lumen 102 of the access sheath 100. In some embodiments, the method of treating the kidney 30 of the patient may include releasably securing the bridge 130 to the proximal end 104 and/or the proximal hub 108 of the access sheath 100 such that the lumen or multiple lumens (e.g., the first lumen 132, the second lumen 134, etc.) of the bridge 130 is/are in fluid communication with the lumen 102 of the access sheath 100.

The medical system may include a flexible ureteroscope 160 including a handle 162 and an elongate shaft 164 extending distally from the handle 162. The elongate shaft 164 of the flexible ureteroscope 160 may include a deflectable distal tip 166 and the elongate shaft 164 may have a length of about 26.5 inches (about 67.3 centimeters). In some embodiments, the elongate shaft 164 may have a length of about 15 inches (about 38.1 centimeters), about 20 inches (about 50.8 centimeters), about 25 inches (about 63.5 centimeters), about 28 inches (about 71.1 centimeters), about 30 inches (about 76.2 centimeters), about 35 inches (about 88.9 centimeters), etc. The elongate shaft 164 may be configured to be slidably disposed within the lumen 102 of the access sheath 100. In some embodiments, the elongate shaft 164 may be configured to be slidably disposed within the lumen or lumens (e.g., the first lumen 132, the second lumen 134, etc.) of the bridge 130. In some embodiments, the elongate shaft 164 may be configured to be slidably disposed within the lumen 102 of the access sheath 100 and the lumen or lumens (e.g., the first lumen 132, the second lumen 134, etc.) of the bridge 130.

The handle 162 of the flexible ureteroscope 160 may include a distal end, a proximal end, and a medial region positioned between the distal end and the proximal end. In general, the elongate shaft 164 may take the form of a polymer or metal tube. In some embodiments, the elongate shaft 164 may be constructed with a reinforcing braid, liner, web, weave, etc. The elongate shaft 164 may include a lumen defining a working channel extending through the elongate shaft 164 from a distal end region of the elongate shaft 164 to an access port 168 at and/or connected to the handle 162. In some embodiments, the elongate shaft 164 may include multiple working channels, as desired.

The deflectable distal tip 166 of the elongate shaft 164 of the flexible ureteroscope 160 may include optics and illumination means disposed therein. The optics and illumination means may be configured to provide optical visualization of the area of the patient being treated and/or traversed by the deflectable distal tip 166. The handle 162 of the flexible ureteroscope 160 may include an electronics and/or optics connector 175 configured to connect the optics and illumination means of the flexible ureteroscope 160 to a controller, a monitor, a display, a computer, etc.

The handle 162 may include a deflection knob 170, which may be used to control movement of the deflectable distal tip 166 of the elongate shaft 164 during operation. For example, the deflection knob 170 may control up and down movement or deflection of the deflectable distal tip 166 of the elongate shaft 164. The deflection knob 170 may be a self-locking or friction lock type knob which maintains the deflection knob 170 and/or the deflectable distal tip 166 at its deflected position after being released. The handle 162 may also include one or a plurality of buttons 172, which may be used to activate irrigation and/or aspiration of a fluid such as air, saline, water, and/or blood, etc. through a lumen of the flexible ureteroscope 160 or perform other functions as desired. These are just examples. Other configurations, variations, and/or features for the flexible ureteroscope 160 are also contemplated.

In some embodiments, the handle 162 may include an aperture 178 disposed at and/or adjacent to the proximal end of the handle 162. In some embodiments, the aperture 178 may be integrally formed into the handle 162. In some embodiments, the aperture 178 may be fixedly and/or permanently attached to the proximal end of the handle 162. In some embodiments, the handle 162 may be suspended using the aperture 178. For example, the handle 162 could be hung on an IV pole, the patient's drapes, the practitioner's gown, a lanyard or strap that may be hung around the practitioner's neck, a mechanical arm, etc. using a hook, a loop, an elastic cord, a screw, a clip, and the like. In one example, the handle 162 may be hung using a retractable lanyard having a constant force spring that counterbalances the weight of the handle 162 of the flexible ureteroscope 160 such that the retractable lanyard and the handle 162 remain in a balanced state to maintain a suspended position of the handle 162 when released. This may reduce the weight of the handle 162 felt by the practitioner during use. In another example, the handle 162 may be hung using a retractable lanyard having a ratcheting type locking mechanism.

In some embodiments, the method of treating the kidney 30 of the patient may include disposing the flexible ureteroscope 160 and/or the elongate shaft 164 of the flexible ureteroscope 160 within the lumen 102 of the access sheath 100. In some embodiments, the method of treating the kidney 30 of the patient may include advancing the deflectable distal tip 166 of the elongate shaft 164 of the flexible ureteroscope 160 into the lumen of the access sheath 100. In some embodiments, the method of treating the kidney 30 of the patient may include positioning the deflectable distal tip 166 of the elongate shaft 164 of the flexible ureteroscope 160 at and/or adjacent to the distal opening 106 of the access sheath 100.

In some embodiments, the elongate shaft 164 of the flexible ureteroscope 160 may include at least one crosshair marking 174 or other indicia disposed on an outer surface of the elongate shaft 164 and configured to position the deflectable distal tip 166 at the distal opening 106 of the access sheath 100. In some embodiments, the at least one crosshair marking 174 may be configured to rotationally position and/or orient the deflectable distal tip 166 relative to the distal opening 106 of the access sheath 100. In some embodiments, the at least one crosshair marking 174 may be configured to longitudinally position the deflectable distal tip 166 relative to the distal opening 106 of the access sheath 100. In some embodiments, a corresponding crosshair marking 176 or other indicia may be disposed on the proximal hub 108 of the access sheath 100, on the bridge 130, and/or on one or more other medical devices (as described herein) usable in conjunction with the flexible ureteroscope 160 to facilitate positioning and/or orientation of the deflectable distal tip 166 of the elongate shaft 164 of the flexible ureteroscope 160 relative to the distal opening 106 of the access sheath 100.

For example, in some embodiments, the medical system may further comprise an access sheath 150, wherein the access sheath 150 is configured to be slidably disposed within the lumen 102 of the access sheath 100 simultaneously with the elongate shaft 164 of the flexible ureteroscope 160. In some embodiments, the access sheath 150 may be disposed over the elongate shaft 164 of the flexible ureteroscope 160 and/or the elongate shaft 164 of the flexible ureteroscope 160 may be disposed within a lumen of the access sheath 150. In some embodiments, the access sheath 150 may include a Tuohy-Borst connector 152 disposed at a proximal end of the access sheath 150. In some embodiments, the Tuohy-Borst connector 152 may include the corresponding crosshair marking 176 disposed thereon, as shown in FIG. 7.

In an alternative configuration, the access sheath 150 may be disposed alongside the elongate shaft 164 of the flexible ureteroscope 160 within the lumen 102 of the access sheath 100. For example, the access sheath 150 may be disposed within the first lumen 132 of the bridge 130 and the elongate shaft 164 of the flexible ureteroscope 160 may be disposed within the second lumen 134 of the bridge 130, and both the access sheath 150 and the elongate shaft 164 may extend from the bridge 130 into the lumen 102 of the access sheath 100. In another example, the access sheath 150 may be disposed within the second lumen 134 of the bridge 130 and the elongate shaft 164 of the flexible ureteroscope 160 may be disposed within the first lumen 132 of the bridge 130, and both the access sheath 150 and the elongate shaft 164 may extend from the bridge 130 into the lumen 102 of the access sheath 100. The first lumen 132 and the second lumen 134 may be sized according to the intended usage and/or the medical device intended to be slidably disposed therein.

In some embodiments, the method of treating the kidney 30 of the patient may include disposing the flexible ureteroscope 160 within the lumen 102 of the access sheath 100 before advancing the access sheath 100 through the urethra 42 into the bladder 40 of the patient. Doing so may permit the practitioner to utilize the optics of the flexible ureteroscope 160 to guide and/or advance the access sheath 100 through the urethra 42 into the bladder 40. This may be advantageous when advancing the access sheath 100 through the urethra 42 of a male patient. In some embodiments, the method of treating the kidney 30 of the patient may include disposing the flexible ureteroscope 160 within the lumen 102 of the access sheath 100 before advancing the access sheath 100 through the urethra 42 of a female patient, as well. Use of the optics already present in the flexible ureteroscope 160 avoids duplication of equipment and function during the procedure and facilitates a reduction in procedure cost.

In some embodiments, disposing the elongate shaft 164 of the flexible ureteroscope 160 within the lumen 102 of the access sheath 100 may include aligning (longitudinally and/or rotationally) at least one crosshair marking 174 on the elongate shaft 164 of the flexible ureteroscope 160 with the corresponding crosshair marking 176 on the proximal hub 108 of the access sheath 100, on the bridge 130, on the Tuohy-Borst connector 152 of the access sheath 150, and/or on another medical device used herewith and locking an orientation of the elongate shaft 164 of the flexible ureteroscope 160 longitudinally and/or rotationally relative to the access sheath 100, the bridge 130, the access sheath 150, etc.

In some embodiments, when the deflectable distal tip 166 of the elongate shaft 164 is positioned at the distal opening 106 of the access sheath 100, at least 10% of the length of the elongate shaft 164 extends proximal of the access sheath 100, the proximal hub 108, and/or the bridge 130. In some embodiments, when the deflectable distal tip 166 of the elongate shaft 164 is positioned at the distal opening 106 of the access sheath 100, at least 20% of the length of the elongate shaft 164 extends proximal of the access sheath 100, the proximal hub 108, and/or the bridge 130. In some embodiments, when the deflectable distal tip 166 of the elongate shaft 164 is positioned at the distal opening 106 of the access sheath 100, at least 30% of the length of the elongate shaft 164 extends proximal of the access sheath 100, the proximal hub 108, and/or the bridge 130. In some embodiments, when the deflectable distal tip 166 of the elongate shaft 164 is positioned at the distal opening 106 of the access sheath 100, at least 40% of the length of the elongate shaft 164 extends proximal of the access sheath 100, the proximal hub 108, and/or the bridge 130.

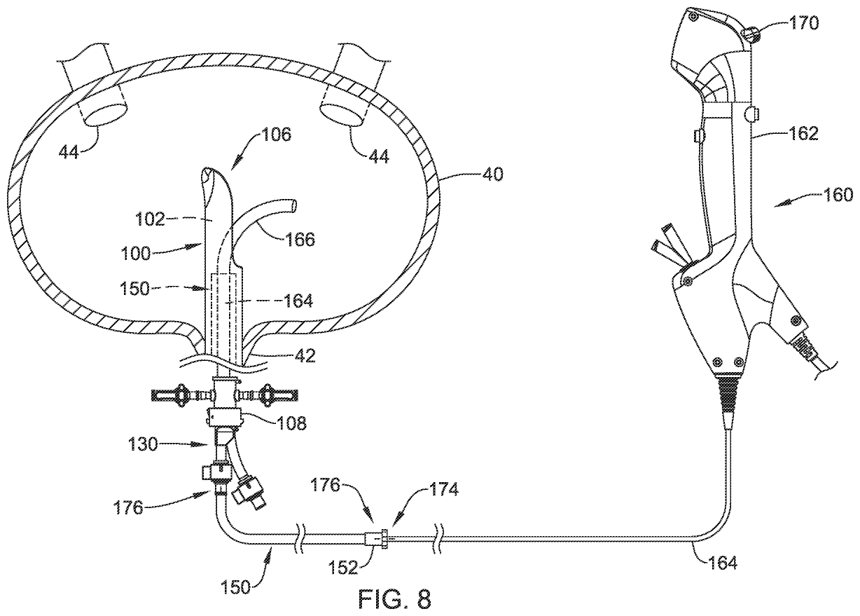

In some embodiments, while the deflectable distal tip 166 may be disposed within and protected by the access sheath 100, the deflectable distal tip 166 may be configured to deflect through the distal opening 106 to provide visualization (e.g., illumination and imaging), of the bladder 40 for example. In some embodiments, the deflectable distal tip 166 may be configured to deflect within a deflection plane (e.g., in a planar manner) perpendicular to the central longitudinal axis of the elongate shaft 164 of the flexible ureteroscope 160. Aligning the at least one crosshair marking 174 on the elongate shaft 164 of the flexible ureteroscope 160 with the corresponding crosshair marking 176 on the proximal hub 108 of the access sheath 100, on the bridge 130, on the Tuohy-Borst connector 152 of the access sheath 150, and/or on another medical device used herewith may rotationally align and/or orient the deflection plane with the distal opening 106 of the access sheath 100, which may extend proximally from the distal tip of the access sheath 100, thus providing passage for the deflectable distal tip 166 to an exterior of the access sheath 100 when actuated by the deflection knob 170, as seen in FIG. 8.

In some embodiments, the method of treating the kidney 30 of the patient may include deflecting the deflectable distal tip 166 through the distal opening 106 of the access sheath 100. In some embodiments, the method of treating the kidney 30 of the patient may include visually locating a ureteric orifice 44 within the bladder 40 using the deflectable distal tip 166 of the elongate shaft 164 of the flexible ureteroscope 160 while the elongate shaft 164 of the flexible ureteroscope 160 is disposed within the lumen 102 of the access sheath 100. Using the deflectable distal tip 166 of the flexible ureteroscope 160 permits greater flexibility and greater field of view than using the fixed optics found in the cystoscope 10 (e.g., FIG. 1).

The method of treating the kidney 30 of the patient may include inserting a guidewire 158 into a working lumen of the flexible ureteroscope 160. In some embodiments, the guidewire may be inserted into the working lumen of the flexible ureteroscope before deflecting the deflectable distal tip 166. In some embodiments, the guidewire may be inserted into the working lumen of the flexible ureteroscope after deflecting the deflectable distal tip 166 and/or after visually locating the ureteric orifice 44.

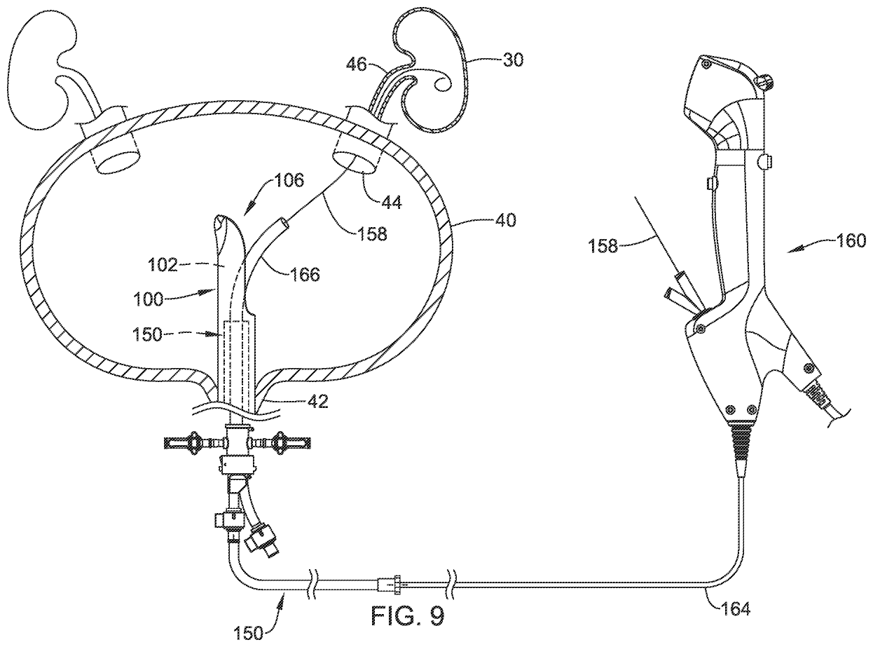

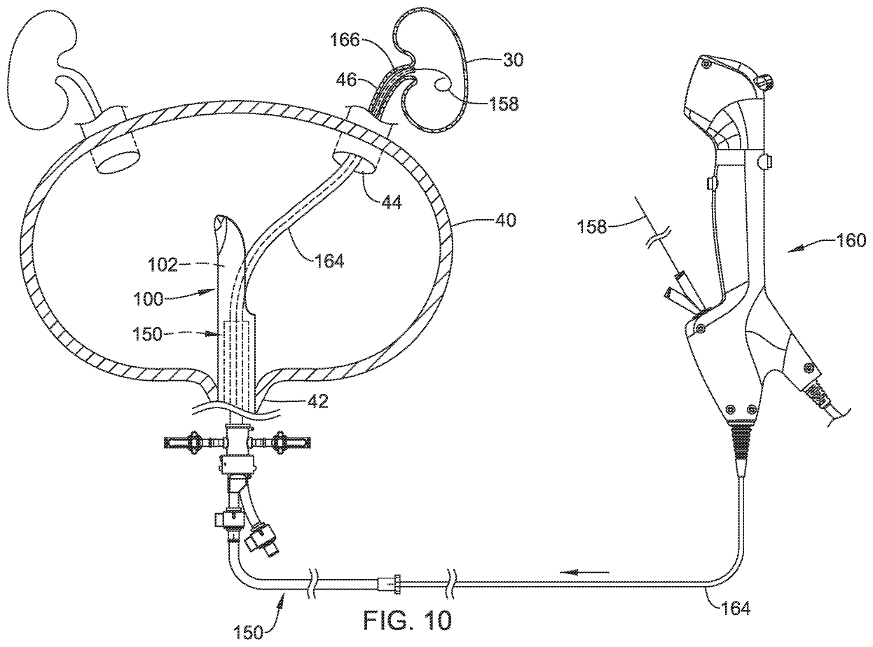

The method of treating the kidney 30 of the patient may include advancing the guidewire 158 through the working channel of the flexible ureteroscope 160 into the ureteric orifice 44 and, if possible, through the ureter 46 to the kidney 30, as shown in FIG. 9. After advancing the guidewire into the ureteric orifice 44 and/or the kidney 30, the method of treating the kidney 30 of the patient may include advancing the deflectable distal tip 166 and/or the elongate shaft 164 of the flexible ureteroscope 160 through the ureteric orifice 44 and to or into the kidney 30 while the elongate shaft 164 of the flexible ureteroscope 160 is disposed within the lumen 102 of the access sheath 100, as shown in FIG. 10. In some embodiments, the method of treating the kidney 30 of the patient may include performing a procedure within the kidney 30 using the flexible ureteroscope 160 while the elongate shaft 164 of the flexible ureteroscope 160 is disposed within the lumen 102 of the access sheath 100. In one example, the flexible ureteroscope 160 may be used to identify and/or find a renal stone, which may be pulverized and/or removed using the flexible ureteroscope 160 and/or another medical device (e.g., a laser fiber, a retrieval net, a forceps, an immobilization device, a stone sweeping device, etc.) disposed within and/or through the working lumen of the flexible ureteroscope 160.

In some embodiments, the method of treating the kidney 30 of the patient may include disposing an access sheath 150 over the guidewire 158. In some embodiments, the method of treating the kidney 30 of the patient may include disposing the access sheath 150 over the elongate shaft 164 of the flexible ureteroscope 160. In some embodiments, disposing the access sheath 150 over the guidewire 158 may include disposing the access sheath 150 over the elongate shaft 164 of the flexible ureteroscope 160. In some embodiments, the access sheath 150 may be disposed over the elongate shaft 164 of the flexible ureteroscope 160 before inserting the guidewire 158 into the working lumen of the flexible ureteroscope 160, as seen in FIGS. 7-9. In some embodiments, disposing the access sheath 150 over the guidewire 158 may include advancing the guidewire 158 through the working lumen of the flexible ureteroscope 160 and the lumen of the access sheath 150 disposed about the elongate shaft 164 of the flexible ureteroscope 160.

In some embodiments, the method of treating the kidney 30 of the patient may include holding the access sheath 150 stationary relative to the access sheath 100 as the guidewire 158 is advanced into the ureteric orifice 44 and/or the kidney 30. In some embodiments, the method of treating the kidney 30 of the patient may include holding the access sheath 150 stationary relative to the access sheath 100 as the deflectable distal tip 166 and/or the elongate shaft 164 of the flexible ureteroscope 160 is advanced into the ureteric orifice 44 and/or the kidney 30, as seen in FIG. 10.

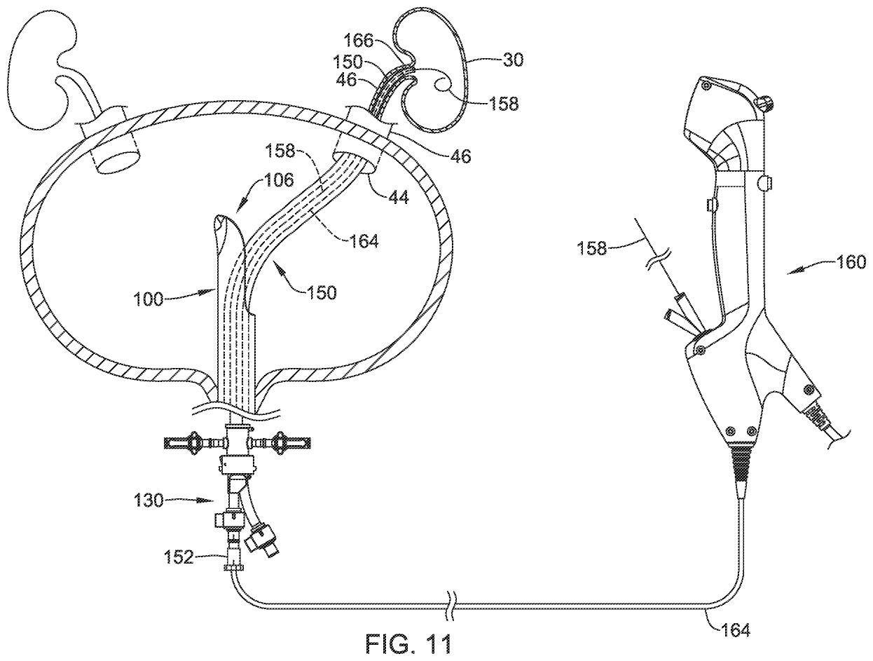

In some embodiments, the method of treating the kidney 30 of the patient may include advancing the access sheath 150 into the ureteric orifice 44 while the flexible ureteroscope 160 is disposed within the lumen 102 of the access sheath 100. In some embodiments, the method of treating the kidney 30 of the patient may include advancing the access sheath 150 over the elongate shaft 164 of the flexible ureteroscope 160 into the ureteric orifice 44 after advancing the deflectable distal tip 166 and/or the elongate shaft 164 of the flexible ureteroscope 160 through the ureteric orifice 44 and into the kidney 30 while the flexible ureteroscope 160 is disposed within the lumen 102 of the access sheath 100, as shown in FIG. 11. In some embodiments, the access sheath 150 may be advanced to a position wherein the Tuohy-Borst connector 152 of the access sheath 150 is disposed at, adjacent to, and/or against the bridge 130. In some embodiments, the access sheath 150 may be advanced to a position wherein a distal end of the access sheath 150 is disposed at, adjacent to, and/or within the kidney 30. In some embodiments, the access sheath 150 may be advanced to a position wherein the distal end of the access sheath 150 is disposed within the ureter 46.

In some embodiments, the method of treating the kidney 30 of the patient may include removing the flexible ureteroscope 160 from the lumen 102 of the access sheath 100 while maintaining the distal end of the guidewire 158 within the kidney 30 and/or the ureter 46. In some embodiments, the method of treating the kidney 30 of the patient may include, after removing the flexible ureteroscope 160 from the lumen 102 of the access sheath 100, advancing the deflectable distal tip 166 of the flexible ureteroscope 160 through the lumen 102 of the access sheath 100 alongside the guidewire 158 and into the bladder 40, as seen in FIG. 12. For example, in some embodiments, the guidewire 158 may be maintained within the first lumen 132 of the bridge 130 and the lumen 102 of the access sheath 100, and the flexible ureteroscope 160 may be inserted into and/or advanced through the second lumen 134 of the bridge 130 and into the lumen 102 of the access sheath 100 alongside the guidewire 158. In some embodiments, the access sheath 150 may be removed from the lumen 102 of the access sheath 100 along with the flexible ureteroscope 160 and then discarded. In some embodiments, the access sheath 150 may be maintained in position along with the guidewire 158 such that the access sheath 150 extends into the ureter 46 and/or the kidney 30 after the flexible ureteroscope 160 has been removed.

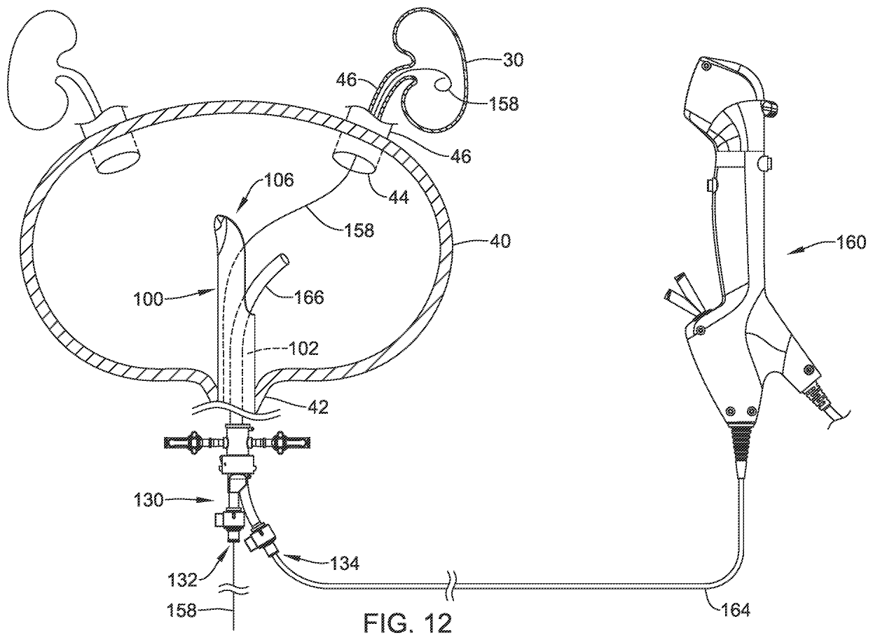

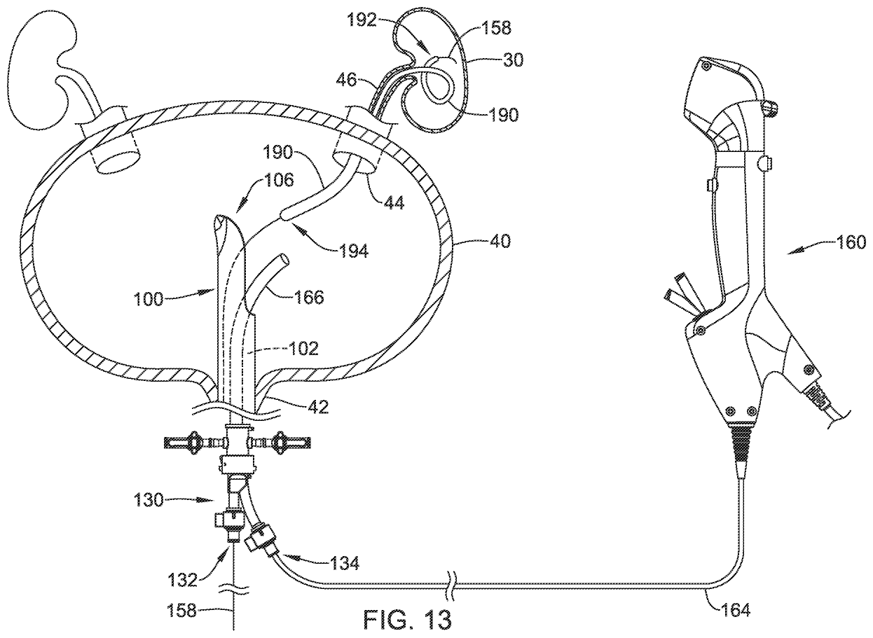

In some embodiments, the method of treating the kidney 30 of the patient may include advancing a stent 190 over the guidewire 158 while the elongate shaft 164 of the flexible ureteroscope 160 is disposed within the lumen 102 of the access sheath 100. In some embodiments, the stent 190 may be advanced over the guidewire 158 and within the access sheath 150 while the elongate shaft 164 of the flexible ureteroscope 160 is disposed within the lumen 102 of the access sheath 100. The method of treating the kidney 30 of the patient may further include positioning a distal end 192 of the stent 190 within the kidney 30 while the elongate shaft 164 of the flexible ureteroscope 160 is disposed within the lumen 102 of the access sheath 100, as shown in FIG. 13. Thereafter, the guidewire 158 and/or the access sheath 150 may be removed from the ureter 46 and the bladder 40 through the lumen 102 of the access sheath 100. A proximal end 194 of the stent 190 may be disposed and/or positioned within the bladder 40. For example, the stent 190 may be a tubular structure disposed within the ureter 46 and configured to transport fluid (e.g., urine) from the kidney 30 to the bladder 40. In some embodiments, the method of treating the kidney 30 of the patient may include visually confirming placement of the stent 190 using the optics of the flexible ureteroscope 160 while the elongate shaft 164 of the flexible ureteroscope 160 is disposed within the lumen 102 of the access sheath 100.

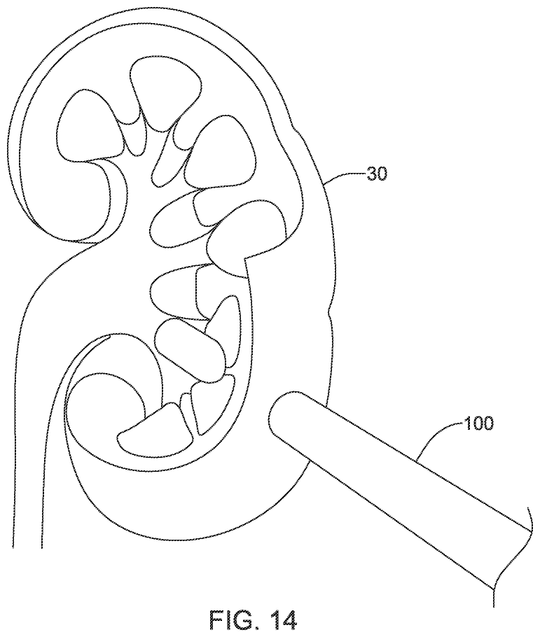

FIGS. 14 and 15 illustrate additional aspects of the medical system in use for treating the urinary tract of a patient in an antegrade approach. In such a method, the kidney 30 of the patient may be accessed through a skin incision or puncture site. For, example, the clinician may use a technique similar to a PCNL procedure to access the urinary tract via the kidney 30. For instance, the clinician may initially pass a needle through a puncture site on the posterior of the patient to the kidney 30. A guidewire may then be advanced through the lumen of the needle such that the distal tip of the guidewire is positioned in the interior of the kidney 30. Thereafter, the needle may be withdrawn, leaving the guidewire in position to maintain access to the kidney 30. After removing the needle, a series of dilators may be advanced over the guidewire and/or a previously positioned dilator to dilate an access path to the kidney 30. Once sufficiently dilated, an access sheath, such as the rigid access sheath 100, may be advanced through the puncture site to the interior of the kidney 30. For example, as shown in FIG. 14, the access sheath 100 may be advanced to the kidney 30 through the puncture site such that the distal end of the access sheath 100 may be positioned in a minor calyx, a major calyx, or renal pelvis of the kidney 30.

Once the access sheath 100 is positioned, the elongate shaft 164 of the flexible ureteroscope 160, or other flexible endoscopic device, may be advanced through the lumen of the access sheath 100 into the kidney 30. The elongate shaft 164 may be advanced beyond the distal tip of the access sheath 100 and then deflected or otherwise navigated to advance through the renal pelvis and into the ureter 41 to a treatment site in the ureter 41. If desired, the elongate shaft 164 may be further advanced through the ureter 41 to a treatment site in the bladder 40 and/or the urethra 42 of the patient. Thus, the elongate shaft 164 of the ureteroscope 160 may have a sufficient length to extend from the proximal end of the access sheath 100, positioned at the puncture site on the posterior of the patient, into the kidney 30, through the ureter 41, and into the bladder 40 and/or urethra 42 of the patient in an antegrade pathway.

Other surgical procedures of the urinary tract that may utilize the access sheath 100 and/or ureteroscope 160, or other medical devices disclosed herein, may include benign prostatic hyperplasia (BPH). In such medical procedures, the access sheath 100 and the ureteroscope 160 may be used to access the patient's prostate through the patient's urethra. For example, the flexible ureteroscope 160 may be passed into the patient's urethra using an antegrade or retrograde approach, described herein, and an instrument (e.g. a laser fiber or resection tool) may be advanced through the lumen of the flexible ureteroscope 160 to ablate or resect obstructed prostate tissue.

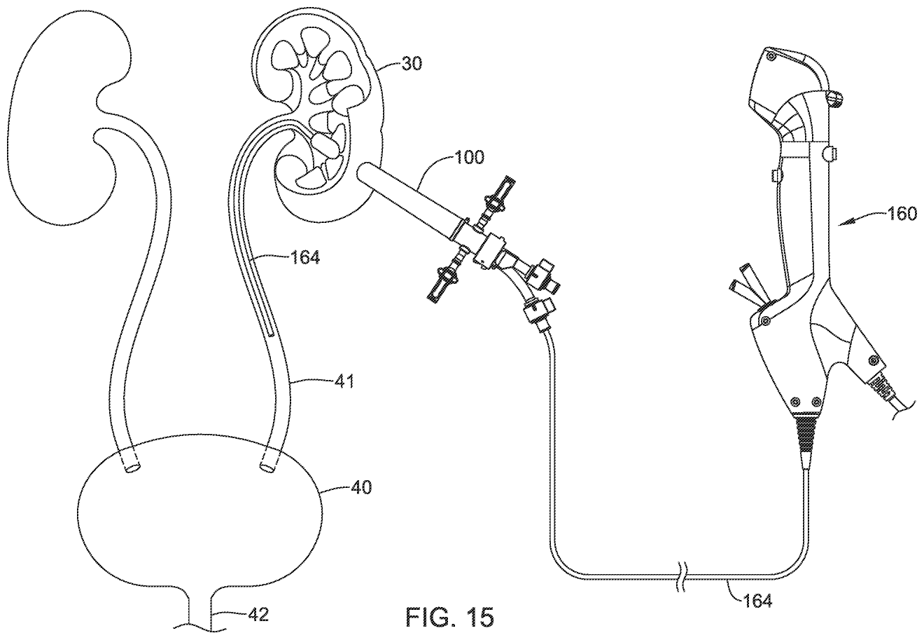

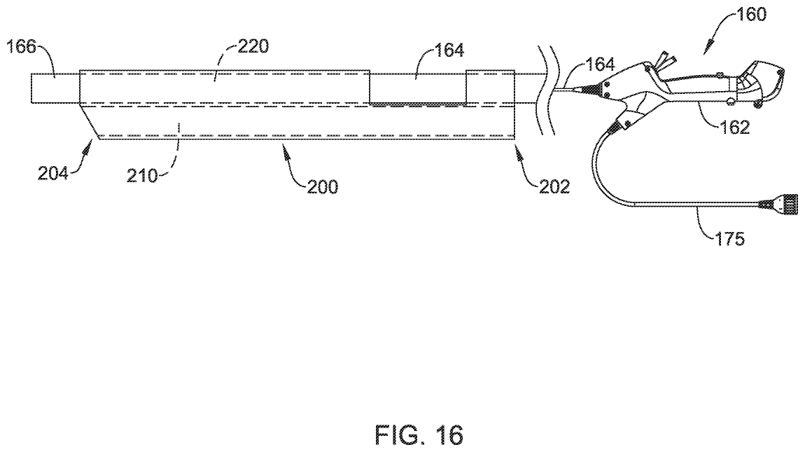

FIG. 16 illustrates aspects of a medical system for treating an anatomical site of a patient, such as a bladder. The medical system may include a flexible endoscopic device, illustrated as the flexible ureteroscope 160 as described above and herein. It is noted that although described as a ureteroscope, the flexible endoscopic device may be designated for other anatomical treatment or diagnostics, such as a nephroscope, bronchoscope, hysteroscope, resectoscope, cystoscope, colonoscope, duodenoscope, esophagoscope, etc. The endoscopic device, referred to herein as the flexible ureteroscope 160, may include the elongate shaft 164 having the deflectable distal tip 166, a working lumen extending therethrough, and optics and illumination means disposed within the deflectable distal tip 166 and in electronic communication with the handle 162 of the flexible ureteroscope 160. The handle 162 of the flexible ureteroscope 160 may include an electronics and/or optics connector 175 configured to connect the optics and illumination means of the flexible ureteroscope 160 to a controller, a monitor, a display, a computer, etc. Other aspects, elements, and/or features of the flexible ureteroscope 160 may be as described above and herein.

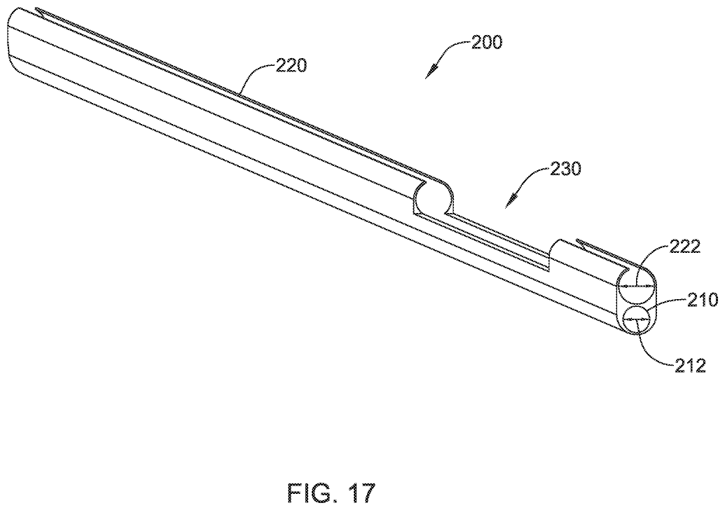

The medical system may include an elongate adapter element 200 configured to removably attach to the elongate shaft 164 of the flexible endoscopic device (e.g., flexible ureteroscope 160) as seen in FIG. 16. The elongate adapter element 200 may include a working channel 210 extending from a proximal end 202 of the elongate adapter element 200 to a distal end 204 of the elongate adapter element 200. In some embodiments, the elongate adapter element 200 may include an open slot 220 extending along a majority of a length of the elongate adapter element 200, as seen in FIG. 17. In some embodiments, the open slot 220 may extend along an entirety of the length of the elongate adapter element 200. Other configurations are also contemplated. In at least some embodiments, a central longitudinal axis of the working channel 210 of the elongate adapter element 200 may be oriented parallel to a central longitudinal axis of the open slot 220.

The working channel 210 may be circumferentially enclosed from the proximal end 202 of the elongate adapter element 200 to the distal end 204 of the elongate adapter element 200. The working channel 210 may have a first cross-sectional area 212. In at least some embodiments, the working channel 210 may have a constant inner extent and/or cross-sectional area along its entire length. In some embodiments, the working channel 210 may be tapered and/or the first cross-sectional area 212 may vary along the length of the working channel 210. In some embodiments, the open slot 220 may have a second cross-sectional area 222 greater than the first cross-sectional area 212. In some embodiments, the second cross-sectional area 222 may be substantially equal to the first cross-sectional area 212. Other configurations are also contemplated. In some embodiments, the elongate adapter element 200 may include a laterally facing cutout 230 in communication with the open slot 220 and disposed between the proximal end 202 of the elongate adapter element 200 and a longitudinal midpoint of the elongate adapter element 200.

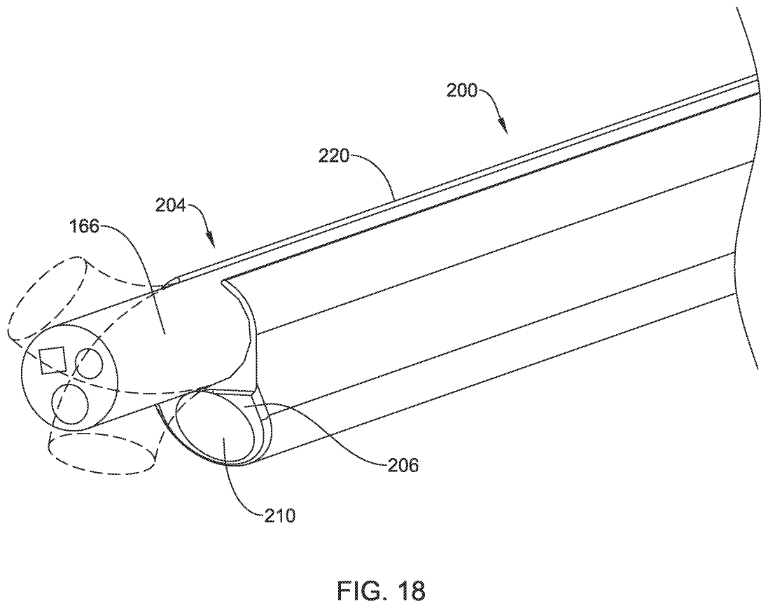

As shown in FIG. 18, when at least a portion of the elongate shaft 164 of the endoscopic device (e.g., the flexible ureteroscope 160) is disposed within the open slot 220, the elongate adapter element 200 may extend circumferentially around at least 50% of a circumference of the elongate shaft 164 of the flexible ureteroscope 160. In some embodiments, when at least a portion of the elongate shaft 164 of the flexible ureteroscope 160 is disposed within the open slot 220, the elongate adapter element 200 may extend circumferentially around at least 60% of a circumference of the elongate shaft 164 of the flexible ureteroscope 160. In some embodiments, when at least a portion of the elongate shaft 164 of the flexible ureteroscope 160 is disposed within the open slot 220, the elongate adapter element 200 may extend circumferentially around at least 70% of a circumference of the elongate shaft 164 of the flexible ureteroscope 160. In some embodiments, when at least a portion of the elongate shaft 164 of the flexible ureteroscope 160 is disposed within the open slot 220, the elongate adapter element 200 may extend circumferentially around at least 80% of a circumference of the elongate shaft 164 of the flexible ureteroscope 160.

In some embodiments, the open slot 220 may be configured to engage the elongate shaft 164 of the endoscopic device (e.g., the flexible ureteroscope 160) with a snap fit, a friction fit, etc. In some embodiments, the elongate adapter element 200 may be configured to apply a radially inward force (e.g., a compressive force) on an outer surface of the elongate shaft 164 of the flexible ureteroscope 160 when at least a portion of the elongate shaft 164 of the flexible ureteroscope 160 is disposed within the open slot 220. In some embodiments, the distal end 204 of the elongate adapter element 200 may be configured to be positioned adjacent to the deflectable distal tip 166 of the elongate shaft 164 of the flexible ureteroscope 160. In at least some embodiments, the distal end 204 of the elongate adapter element 200 may be configured to be positioned proximal of the deflectable distal tip 166 of the elongate shaft 164 of the flexible ureteroscope 160. In some embodiments, the deflectable distal tip 166 may extend about 1 centimeter (cm), about 1.5 cm, about 2 cm, about 2.5 cm, about 3 cm, etc. distal of the distal end 204 of the elongate adapter element 200.

As may be seen in FIG. 18, the deflectable distal tip 166 and/or the elongate shaft 164 of the endoscopic device (e.g., the flexible ureteroscope 160) may include a working channel, optics, and illumination means. The deflectable distal tip 166 may be configured to deflect within a deflection plane (e.g., up and/or down, left and/or right, etc.). In at least some embodiments, the deflection plane may be parallel to and/or coincident with a plane containing the central longitudinal axis of the working channel 210 of the elongate adapter element 200 and the central longitudinal axis of the open slot 220. In some embodiments, the deflection plane may be skewed to the plane containing the central longitudinal axis of the working channel 210 of the elongate adapter element 200 and the central longitudinal axis of the open slot 220. Deflection of the deflectable distal tip 166 may provide increased field of view for the optics and/or an ability to direct a medical device extending through the working channel of the elongate adapter element 200. In some embodiments, a portion of the distal end 204 having the working channel 210 disposed therein may include an angled face 206 to increase range of motion and/or accessibility for a medical device disposed within the working channel 210.

In some embodiments, the elongate adapter element 200 may be substantially rigid along its length. In some embodiments, the elongate adapter element 200 may be semi-rigid along its length. In some embodiments, the elongate adapter element 200 may be formed as an extruded structure. In some embodiments, the elongate adapter element 200 may be formed from a metallic material, a polymeric material, and/or a composite material, etc. In at least some embodiments, the elongate adapter element 200 may be formed from a radiopaque material and/or may be formed with a radiopaque material embedded therein. In some embodiments, the elongate adapter element 200 may include radiopaque markers embedded within the elongate adapter element 200 and/or attached to the elongate adapter element 200. Other configurations are also contemplated.

In some embodiments, the elongate adapter element 200 may include a support member configured to secure excess length of the elongate shaft 164 of the flexible ureteroscope 160 relative to the elongate adapter element 200. The support member may take one or more of several different forms described herein. The described forms may be collectively referred to by the term “support member”.

In some embodiments, when at least a portion of the elongate shaft 164 of the flexible ureteroscope 160 is disposed within the open slot 220, a portion (e.g., excess length) of the elongate shaft 164 of the flexible ureteroscope 160 may extend through the laterally facing cutout 230 and is positioned outside of the elongate adapter element 200 and/or the open slot 220. Depending on the procedure, it may be beneficial to secure the portion (e.g., the excess length) of the elongate shaft 164 of the flexible ureteroscope 160 relative to the handle 162 and/or the elongate adapter element 200, such as with the support member(s) described herein.

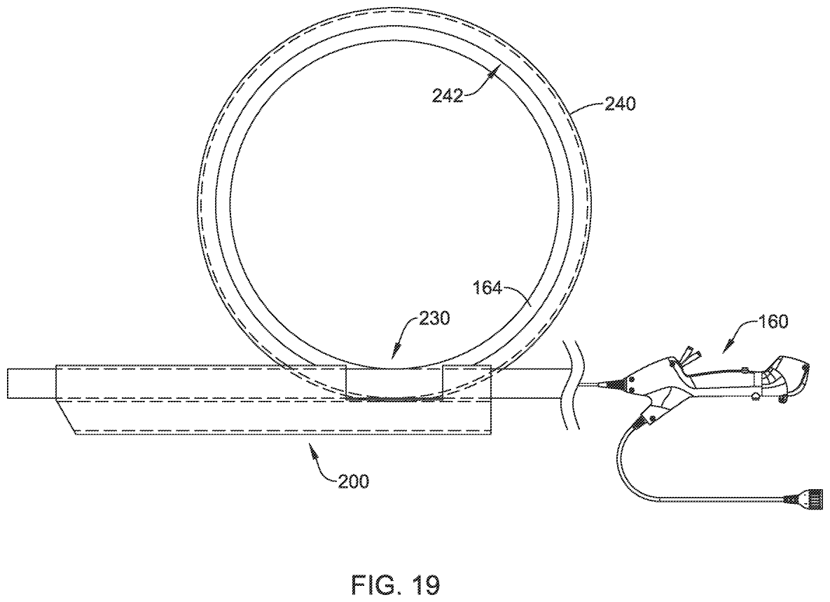

In some embodiments, the support member of the elongate adapter element 200 may include a ring 240, as seen in FIG. 19. In some embodiments, the support member and/or the ring 240 may be configured to form a portion of the length and/or the excess length of the elongate shaft 164 of the flexible ureteroscope 160 into a loop. In some embodiments, the support member and/or the ring 240 may be fixedly attached to the elongate adapter element 200. In some embodiments, the support member and/or the ring 240 may be permanently attached to the elongate adapter element 200. In some embodiments, the support member and/or the ring 240 may be integrally formed with the elongate adapter element 200 as a monolithic structure. In some embodiments, the support member and/or the ring 240 may be removably attached to the elongate adapter element 200.

In some embodiments, the support member and/or the ring 240 may be disposed alongside the open slot 220 and/or the elongate adapter element 200. In some embodiments, the support member and/or the ring 240 may include a circumferentially continuous outer surface. In some embodiments, the support member and/or the ring 240 may include a discontinuous outer surface (e.g., the support member and/or the ring 240 may include apertures, notches, cutouts, etc.). In some embodiments, the support member and/or the ring 240 may have a semi-circular cross-section defining a channel 242 facing radially inwards. The channel 242 may be configured to receive at least a portion of the elongate shaft 164 of the flexible ureteroscope 160 therein.

In some embodiments, when at least a portion of the elongate shaft 164 of the endoscopic device (e.g., the flexible ureteroscope 160) is disposed within the channel 242, the support member and/or the ring 240 may extend circumferentially around at least 30% of a circumference of the elongate shaft 164 of the flexible ureteroscope 160. In some embodiments, when at least a portion of the elongate shaft 164 of the flexible ureteroscope 160 is disposed within the channel 242, the support member and/or the ring 240 may extend circumferentially around at least 40% of the circumference of the elongate shaft 164 of the flexible ureteroscope 160. In some embodiments, when at least a portion of the elongate shaft 164 of the flexible ureteroscope 160 is disposed within the channel 242, the support member and/or the ring 240 may extend circumferentially around at least 50% of the circumference of the elongate shaft 164 of the flexible ureteroscope 160. In some embodiments, when at least a portion of the elongate shaft 164 of the flexible ureteroscope 160 is disposed within the channel 242, the support member and/or the ring 240 may extend circumferentially around at least 60% of the circumference of the elongate shaft 164 of the flexible ureteroscope 160.

In some embodiments, the channel 242 may be configured to engage the elongate shaft 164 of the flexible ureteroscope 160 with a snap fit, a friction fit, etc. In some embodiments, the support member and/or the ring 240 may be configured to apply a radially inward force (e.g., a compressive force) on an outer surface of the elongate shaft 164 of the flexible ureteroscope 160 when at least a portion of the elongate shaft 164 of the flexible ureteroscope 160 is disposed within the channel 242. In some embodiments, the elongate shaft 164 of the flexible ureteroscope 160 may be configured and/or permitted to slide and/or move within the channel 242 when at least a portion of the elongate shaft 164 of the flexible ureteroscope 160 is disposed within the channel 242. Other configurations are also contemplated.

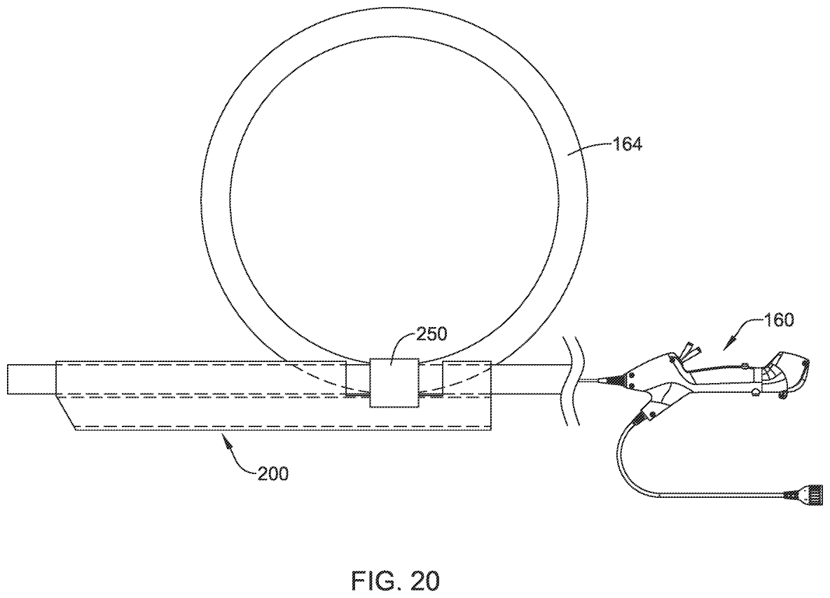

In some embodiments, the support member of the elongate adapter element 200 may include a clip 250, as seen in FIG. 20. In some embodiments, the support member and/or the clip 250 may be configured to form a portion of the length and/or the excess length of the elongate shaft 164 of the flexible ureteroscope 160 into a loop. In some embodiments, the support member and/or the clip 250 may be fixedly attached to the elongate adapter element 200. In some embodiments, the support member and/or the clip 250 may be permanently attached to the elongate adapter element 200. In some embodiments, the support member and/or the clip 250 may be integrally formed with the elongate adapter element 200 as a monolithic structure. In some embodiments, the support member and/or the clip 250 may be removably attached to the elongate adapter element 200.

In some embodiments, the support member and/or the clip 250 may be disposed alongside the open slot 220 and/or the elongate adapter element 200. The clip 250 may be configured to receive at least a portion of the elongate shaft 164 of the flexible ureteroscope 160 therein. In some embodiments, the clip 250 may be configured to engage the elongate shaft 164 of the flexible ureteroscope 160 with a snap fit, a friction fit, etc. In some embodiments, the clip 250 may be a spring clip that is biased or self-biased toward a closed configuration. In some embodiments, the support member and/or the clip 250 may be configured to apply a radially inward force (e.g., a compressive force) on an outer surface of the elongate shaft 164 of the flexible ureteroscope 160 when at least a portion of the elongate shaft 164 of the flexible ureteroscope 160 is disposed within the clip 250. In some embodiments, the elongate shaft 164 of the flexible ureteroscope 160 may be configured and/or permitted to slide and/or move within the clip 250 when at least a portion of the elongate shaft 164 of the flexible ureteroscope 160 is disposed within the clip 250. Other configurations are also contemplated.

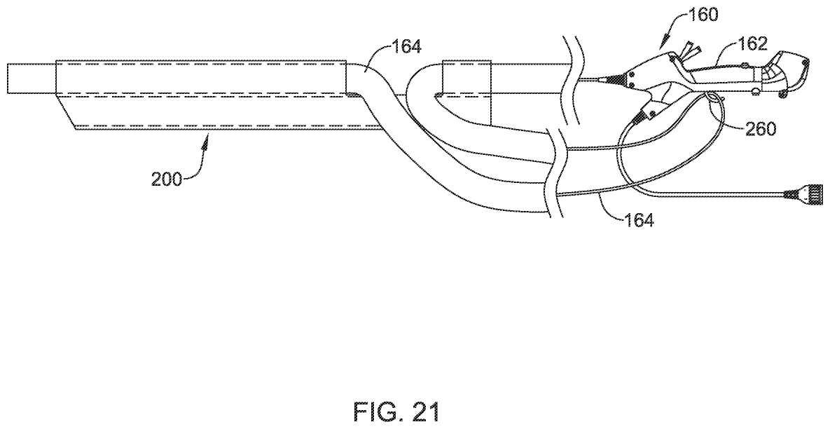

In some embodiments, the support member of the elongate adapter element 200 may include a hook 260. In some embodiments, the support member and/or the hook 260 may be attached to the handle 162 of the flexible ureteroscope 160, as seen in FIG. 21. In some embodiments, the support member and/or the hook 260 may be attached to the elongate adapter element 200. In some embodiments, the support member and/or the hook 260 may be configured to form a portion of the length and/or the excess length of the elongate shaft 164 of the flexible ureteroscope 160 into a loop. In some embodiments, the support member and/or the hook 260 may be fixedly attached to the handle 162 or elongate adapter element 200. In some embodiments, the support member and/or the hook 260 may be permanently attached to the handle 162 or elongate adapter element 200. In some embodiments, the support member and/or the hook 260 may be integrally formed with the handle 162 or elongate adapter element 200 as a monolithic structure. In some embodiments, the support member and/or the hook 260 may be removably attached to the handle 162 or elongate adapter element 200.

The hook 260 may be configured to receive at least a portion of the elongate shaft 164 of the endoscopic device (e.g., the flexible ureteroscope 160) therein. In some embodiments, the hook 260 may be configured to engage the elongate shaft 164 of the flexible ureteroscope 160 with a snap fit, a friction fit, etc. In some embodiments, the support member and/or the hook 260 may be configured to apply a radially inward force (e.g., a compressive force) on an outer surface of the elongate shaft 164 of the flexible ureteroscope 160 when at least a portion of the elongate shaft 164 of the flexible ureteroscope 160 is disposed within the hook 260. In some embodiments, the elongate shaft 164 of the flexible ureteroscope 160 may be configured and/or permitted to slide and/or move within the hook 260 when at least a portion of the elongate shaft 164 of the flexible ureteroscope 160 is disposed within the hook 260. Other configurations are also contemplated.

In some embodiments, the elongate adapter element 200 may further include an attachment portion 270 disposed and/or extending proximal of the proximal end 202. The attachment portion 270 may be configured to removably attach to the distal end of the handle 162 of the flexible ureteroscope 160. The attachment portion 270 may be configured to prevent relative longitudinal movement between the elongate adapter element 200 and the elongate shaft 164 and/or the handle 162 of the flexible ureteroscope 160. In some embodiments, the attachment portion 270 may be fixedly attached to the proximal end 202 of the elongate adapter element 200. In some embodiments, the attachment portion 270 may be integrally formed with the proximal end 202 of the elongate adapter element 200 as a monolithic structure. In some embodiments, the attachment portion 270 may be configured to engage with the distal end of the handle 162 of the flexible ureteroscope 160 via a snap fit, a friction fit, etc. Other configurations are also contemplated. In some embodiments, the attachment portion 270 may comprise a plurality of sections that fit together in an interlocking fashion.

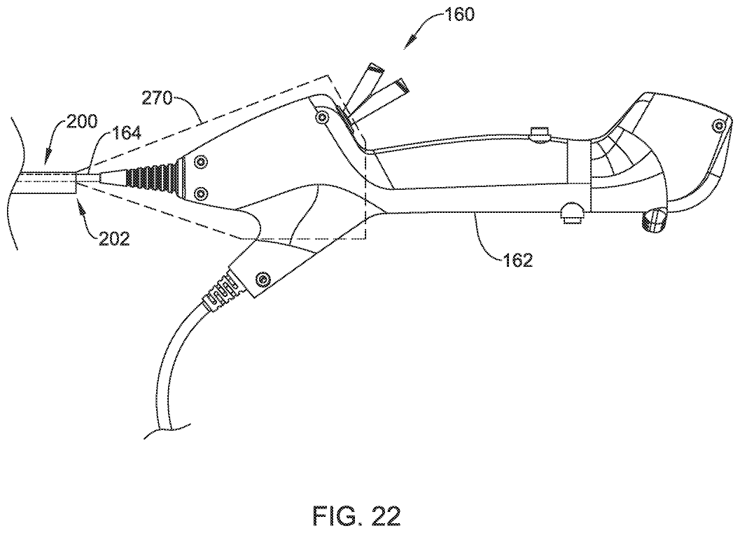

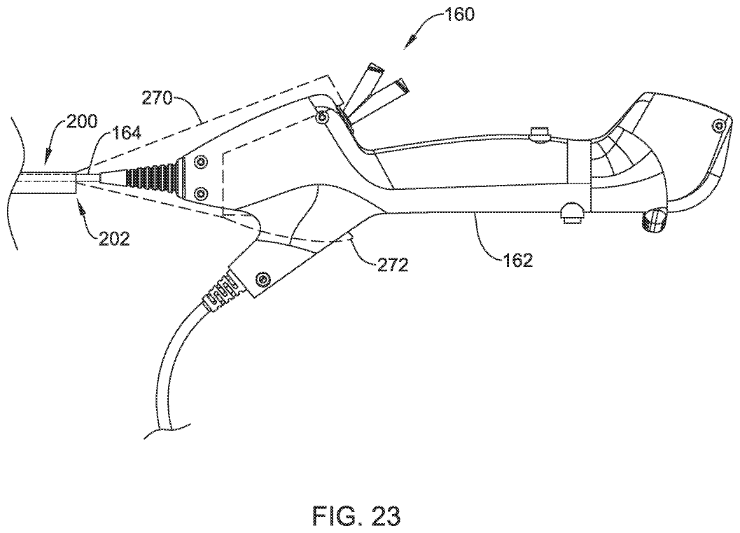

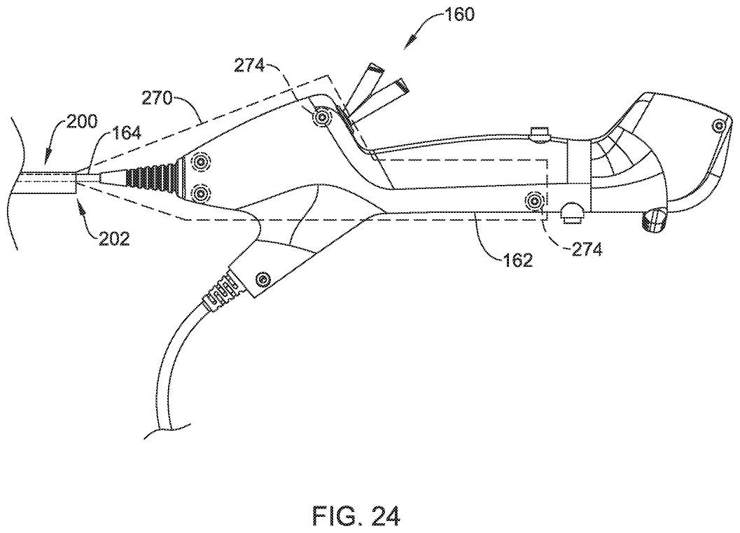

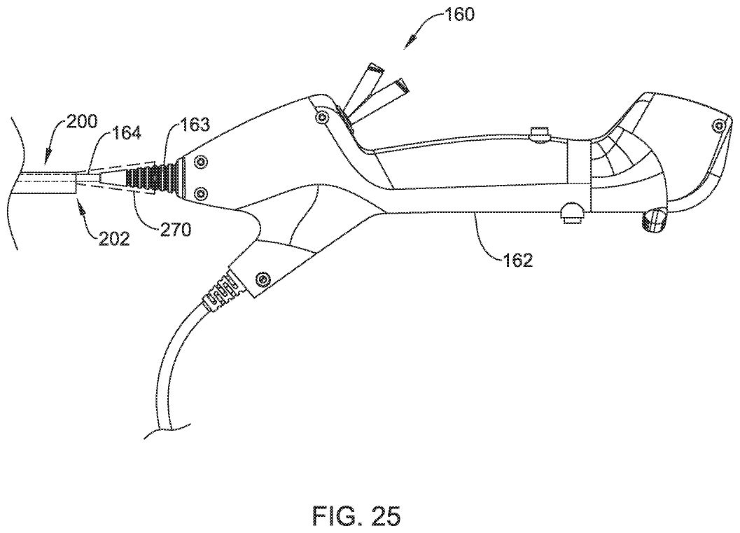

In some embodiments, the attachment portion 270 may be configured to engage and/or cooperate with the distal end of the handle 162 of the flexible ureteroscope 160, as seen in FIG. 22. The attachment portion 270 may include one or more flanges and/or other features configured to wrap around a portion of the distal end of the handle 162 the flexible ureteroscope 160. Contact and/or interference between the attachment portion 270 and/or the handle 162 the flexible ureteroscope 160 may prevent distal movement of the elongate adapter element 200 relative to the elongate shaft 164 and/or the handle 162 of the flexible ureteroscope 160. In some embodiments, the attachment portion 270 may be shaped and/or formed to receive at least a portion of the distal end of the handle 162 of the flexible ureteroscope 160 in a complimentary manner. In some embodiments, the attachment portion 270 may slip over the distal end of the handle 162 of the flexible ureteroscope 160. In some embodiments, the attachment portion 270 may wrap around multiple portions of the handle 162 of the flexible ureteroscope 160. In some embodiments, the attachment portion 270 may include a tether 272 configured to extend around a protruding portion of the handle 162 of the flexible ureteroscope 160, as seen in FIG. 23. In some embodiments, the attachment portion 270 may extend along the medial region of the handle 162 of the flexible ureteroscope 160. In some embodiments, the attachment portion 270 may include a plurality of pins 274 extending inward from the attachment portion 270 toward the handle 162 of the flexible ureteroscope 160. In some embodiments, the plurality of pins 274 may be configured to engage with one or more apertures or holes formed in an outer surface of the handle 162 of the flexible ureteroscope 160, as shown in FIG. 24. In some embodiments, the attachment portion 270 may be configured to engage a strain relief 163 disposed at the distal end of the handle 162 of the flexible ureteroscope 160, as seen in FIG. 25. For example, the attachment portion 270 may be formed in a clam shell configuration configured to extend between adjacent ribs of the strain relief 163. In another example, the attachment portion 270 may be formed with a longitudinally oriented slot along one side such that the attachment portion 270 may slide over the strain relief 163 and a proximal portion of the elongate shaft 164 of the flexible ureteroscope 160 laterally. In yet another example, the attachment portion 270 may include a compression fitting configured to engage the strain relief 163. Other configurations are also contemplated.

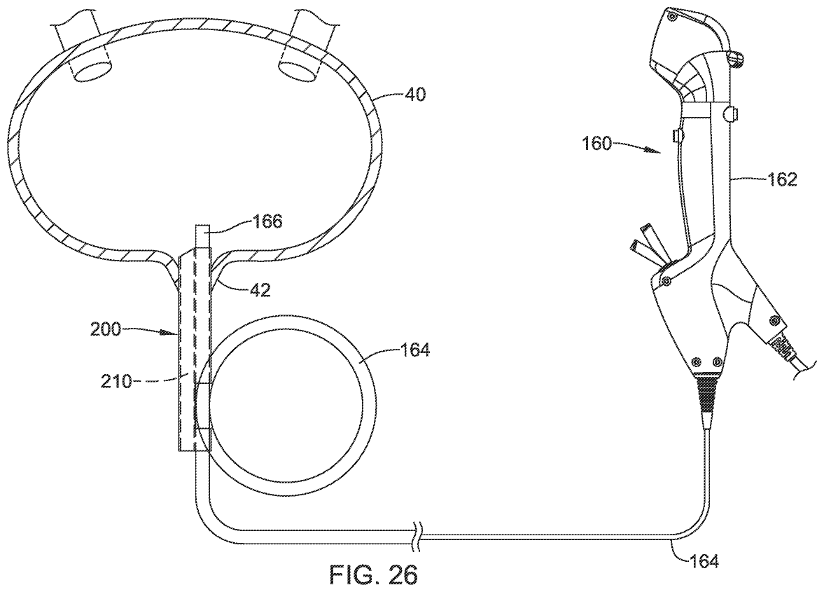

FIG. 26 illustrates an example method for treating the bladder 40 of a patient using the medical system shown in FIGS. 16-25. In some embodiments, the flexible ureteroscope 160 may be longer than necessary for accessing and/or treating the bladder 40 on its own.

The excess length of the elongate shaft 164 of the flexible ureteroscope 160 may interfere with and/or complicate the procedure. The elongate adapter element 200 may be removably attached to a distal portion of the elongate shaft 164 of the flexible ureteroscope 160. In some embodiments, the elongate adapter element 200 may cause the flexible ureteroscope 160 to function similar to the cystoscope 10 for accessing the bladder 40. However, by using the flexible ureteroscope 160 in conjunction with the elongate adapter element 200, the benefits and features of the flexible ureteroscope 160 are retained and may be used during the procedure. For example, the flexible ureteroscope 160 may have higher quality optics then the cystoscope 10. In another example, the deflectable distal tip 166 remains available for use and may permit improved viewing of the interior of the bladder 40 and/or may permit viewing of more of the interior of the bladder 40 compared to the cystoscope 10 without causing discomfort to the patient. The working channel 210 of the elongate adapter element 200 may be larger and/or may have a greater cross-sectional area than the working lumen of the flexible ureteroscope 160, thus permitting larger instruments and/or medical devices to be used when treating the bladder 40 than simply using the flexible ureteroscope 160 alone. Using the flexible ureteroscope 160 in conjunction with the elongate adapter element 200 may also allow the use of two working lumens (e.g., the working channel 210 and the working lumen of the flexible ureteroscope 160) simultaneously.

Additionally, the elongate adapter element 200 may be constructed as a disposable device that may be discarded after use (e.g., a single use device) to reduce and/or eliminate costs associated with disinfection and/or repair. Construction of the elongate adapter element 200 as a disposable device may permit the re-use of the flexible ureteroscope 160 for multiple purposes and/or procedures for a single patient. For example, the elongate adapter element 200 may permit the flexible ureteroscope 160 to function as a cystoscope for accessing and treating the bladder 40. After treating the bladder 40, the elongate adapter element 200 may be removed and/or discarded and the same flexible ureteroscope 160 may be used subsequently during the medical procedure to treat the kidney(s) of the patient, for example. Other configurations and/or uses are also contemplated.

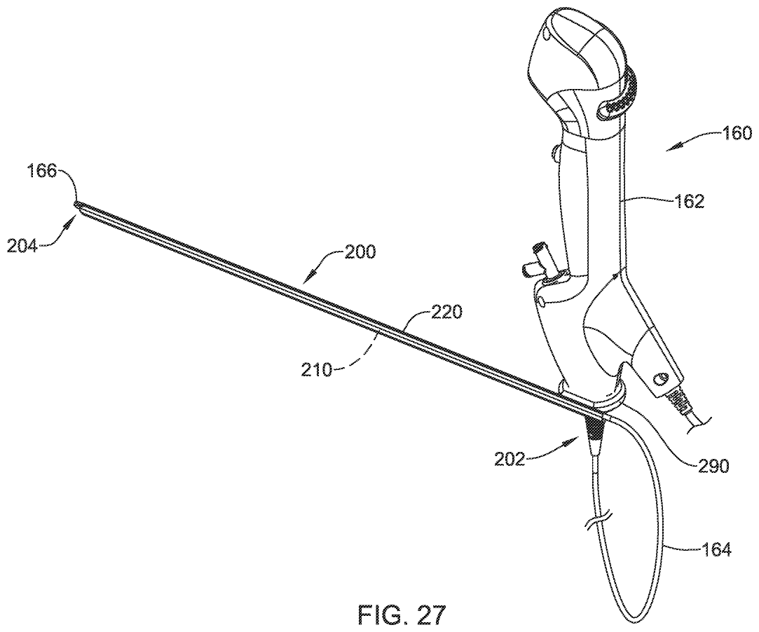

FIG. 27 illustrates an alternative configuration of the elongate adapter element 200. As described herein, the elongate adapter element 200 may include the working channel 210 and the open slot 220. In some embodiments, the elongate adapter element 200 may be devoid of the laterally facing cutout 230. Instead, the elongate adapter element 200 may cause excess length of the elongate shaft 164 of the flexible ureteroscope 160 to be formed into a loop proximal of the elongate adapter element 200 when at least a portion of the elongate shaft 164 of the flexible ureteroscope 160 is disposed within the open slot 220. To do so, the elongate adapter element 200 may include a securement ring 290 attached to a proximal portion of the elongate adapter element 200. The securement ring 290 may be configured to receive the distal end of the handle 162 of the flexible ureteroscope 160 such that the handle 162 of the flexible ureteroscope 160 is oriented at approximately 90 degrees to the elongate adapter element 200. Accordingly, a portion (e.g., the excess length) of the elongate shaft 164 of the flexible ureteroscope 160 disposed proximal of the proximal end 202 of the elongate adapter element 200 may form a loop adjacent to the proximal end 202 of the elongate adapter element 200 and the distal end of the handle 162 of the flexible ureteroscope 160, as shown in FIG. 27.

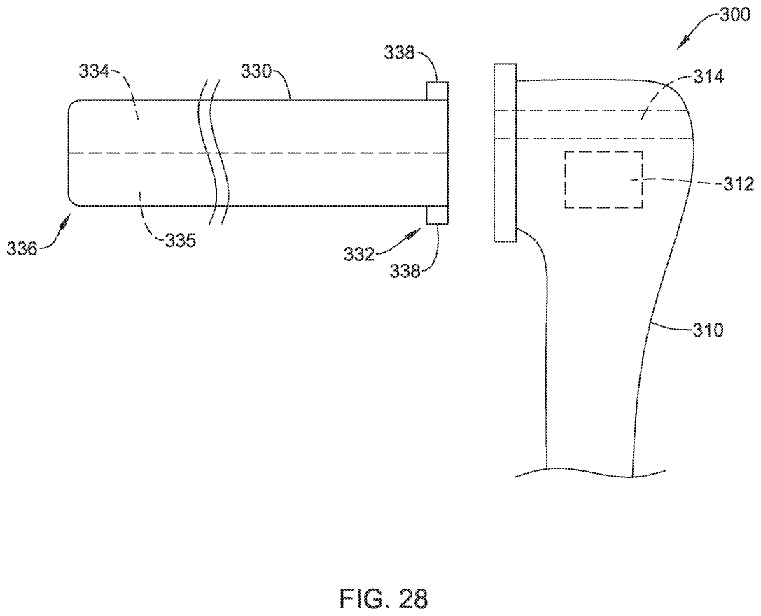

FIG. 28 illustrates aspects of a modular medical device 300. Some medical devices involve expensive components and therefore to recoup the costs of the devices, they are made to be disinfected and reused. However, reusing components that come into contact with a patient's body and/or fluids carries some degree of risk, even with known and/or established disinfection procedures. Accordingly, the modular medical device 300, which includes both reusable and disposable components, is disclosed to reduce costs associated with commonly used medical devices of similar type, such as various endoscope devices (e.g., a ureteroscope (e.g., LithoVue™ scope), nephroscope, bronchoscope, hysteroscope, resectoscope, cystoscope, colonoscope, duodenoscope, esophagoscope, etc.) and other scope devices disclosed herein.

The modular medical device 300 may include a handle portion 310 and a disposable shaft portion 330, wherein a proximal end 332 of the disposable shaft portion 330 may be configured to releasably connect to the handle portion 310. The handle portion 310 may include electronic components 312 configured to communicate with the disposable shaft portion 330 when the proximal end 332 of the disposable shaft portion 330 is connected to the handle portion 310. Since the electronic components 312 are typically some of the most expensive elements in these types of medical devices, and the handle portion 310 typically does not come into direct contact with the patient, the handle portion 310 may be adapted and configured to be reusable through the use of appropriate sterilization and/or maintenance procedures.

In some embodiments, the handle portion 310 may include a proximal working channel 314 extending through the handle portion 310. The handle portion 310 may include a valve or seal associated with the proximal working channel 314 and configured to engage the disposable shaft portion 330 when the proximal end 332 of the disposable shaft portion 330 is connected to the handle portion 310 such that the patient's bodily fluids do not come into contact with the proximal working channel 314. Other methods and mechanisms for reducing and/or preventing contamination of the handle portion 310 are also contemplated. In some embodiments, the proximal working channel 314 may be designed and configured to be easily cleaned and disinfected. In at least some embodiments, the disposable shaft portion 330 may be substantially rigid or semi-rigid along its length. For example, the disposable shaft portion 330 may be made of a metallic material (e.g., stainless steel, etc.), a polymeric material, a composite material, etc. Other configurations are also contemplated.

The disposable shaft portion 330 may include a distal working channel 334 configured to be in fluid communication with the proximal working channel 314 when the disposable shaft portion 330 is connected to the handle portion 310. In some embodiments, the distal working channel 334 may include a backflow preventor or a one-way valve disposed therein to prevent the patient's bodily fluids from reaching and/or contaminating the handle portion 310 and/or the proximal working channel 314. In some embodiments, the disposable shaft portion 330 may include a plurality of locking tabs 338 disposed at and/or proximate the proximal end 332 of the disposable shaft portion 330. The plurality of locking tabs 338 may be configured to releasably engage one or more notches formed on the handle portion 310. In some embodiments, the one or more notches may include a slot. In some embodiments, the disposable shaft portion 330 may be releasably engaged with and/or connected to the handle portion 310 using a twist-lock mechanism. This is just an example. Other configurations are also contemplated. In some embodiments, the disposable shaft portion 330 and/or the handle portion 310 may include a spring element or a plurality of spring elements configured to enhance engagement of the plurality of locking tabs 338 and the one or more notches and/or configured to prevent disengagement of the plurality of locking tabs 338 and the one or more notches. Other configurations are also contemplated.

In some embodiments, the disposable shaft portion 330 may include illumination and imaging components disposed at a distal end 336 of the disposable shaft portion 330. In some embodiments, the illumination and imaging components may be disposed within an elongate sleeve 335 fixedly disposed within the disposable shaft portion 330. The elongate sleeve 335 may be formed of a polymeric material, a metallic material, or other material, as desired. In some embodiments, the elongate sleeve 335 may separate and/or protect the illumination and imaging components from the distal working channel 334 and/or the patient's bodily fluids. In some embodiments, the illumination and imaging components may be in electronic communication with the electronic components 312 of the handle portion 310 when the proximal end 332 of the disposable shaft portion 330 is connected to the handle portion 310. The illumination and imaging components may be configured to illuminate and image an area proximate, near, adjacent to, and/or around the distal end 336 of the disposable shaft portion 330 when the proximal end 332 of the disposable shaft portion 330 is connected to the handle portion 310. In some embodiments, the illumination and imaging components may be fixedly attached to and/or disposed within the distal end 336 of the disposable shaft portion 330. In some embodiments, the illumination and imaging components may be removable from the disposable shaft portion 330 as described herein.

In some embodiments, illumination may be provided through the disposable shaft portion 330 via fiber optics carrying light generated by light emitting diodes (LEDs) disposed within the handle portion 310. In some embodiments, illumination may be provided via LEDs disposed within the handle portion 310. In some embodiments, the LEDs may be disposed at the distal end 336 of the disposable shaft portion 330. In some embodiments, imaging may be provided through the disposable shaft portion 330 via fiber optic bundles extending through the disposable shaft portion 330 which connect to the handle portion 310 and project through an objective lens disposed within the handle portion 310 onto an imager disposed within the handle portion 310. The imager disposed within the handle portion 310 may capture live video images, which may be transmitted to a display, a monitor, a computer, a controller, etc. in electronic communication with the handle portion 310. In some embodiments, the imager may be a complementary metal oxide semiconductor (CMOS) imager.

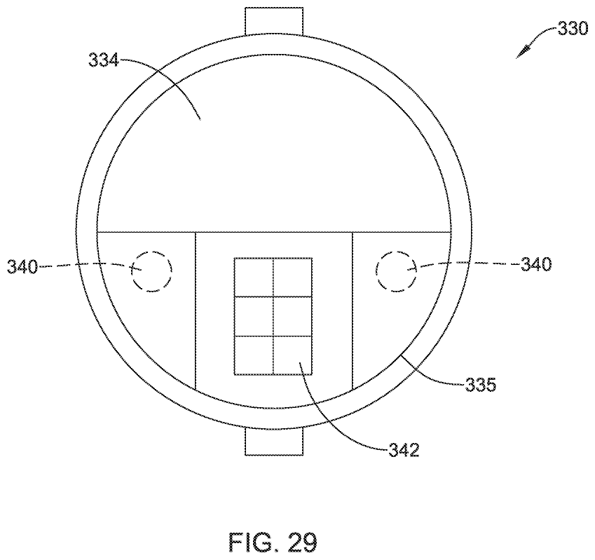

FIG. 29 is a distal end view of one example configuration for the disposable shaft portion 330. Some features described above and called out in the figure (e.g., the plurality of locking tabs 338, etc.) are not repeated in the interest of brevity. In some embodiments, the distal working channel 334 may be disposed within and/or extending along a top portion of the disposable shaft portion 330. In some embodiments, the elongate sleeve 335 may be disposed within and/or extending along a bottom portion of the disposable shaft portion 330. These are just examples. Other configurations are also contemplated. In some embodiments, the disposable shaft portion 330 may optionally include one or more ports 340 disposed at and/or adjacent to the distal end 336. In some embodiments, the one or more ports 340 may be used for fluid delivery and/or removal or other appropriate uses (e.g., a laser fiber, tool delivery, etc.). The one or more ports 340 may be in fluid communication with one or more channels extending along the disposable shaft portion 330. In the illustrated example, fiber optic bundles 342 are shown for illumination and imaging. The fiber optic bundles 342 are shown having a square shape. In some embodiments, the fiber optic bundles 342 may extend about 0.5 millimeters (mm) on each side. This is just an example. Other shapes (e.g., circular, polygonal, rectangular, elliptical, etc.) and/or sizes are also contemplated. In some embodiments, one or more of the fiber optic bundles 342 may align with and/or may be in communication with the imager disposed within the handle portion 310.

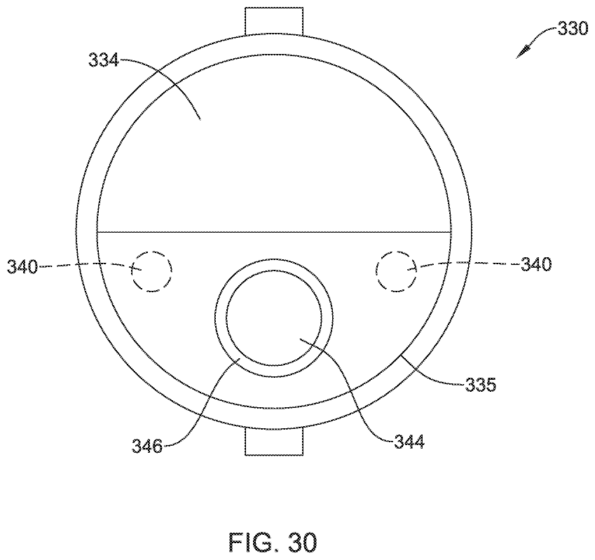

FIG. 30 is a distal end view of another example configuration for the disposable shaft portion 330. Some features described above and called out in the figure (e.g., the plurality of locking tabs 338, etc.) are not repeated in the interest of brevity. In some embodiments, the distal working channel 334 may be disposed within and/or extend along the top portion of the disposable shaft portion 330. In some embodiments, the elongate sleeve 335 may be disposed within and/or extend along a bottom portion of the disposable shaft portion 330. These are just examples. Other configurations are also contemplated. In some embodiments, the disposable shaft portion 330 may optionally include one or more ports 340 disposed at and/or adjacent to the distal end 336. In some embodiments, the one or more ports 340 may be used for fluid delivery and/or removal or other appropriate uses (e.g., a laser fiber, tool delivery, etc.). The one or more ports 340 may be in fluid communication with one or more channels extending along the disposable shaft portion 330. In the illustrated example, a first fiber optic bundle 344 is shown for imaging and a second fiber optic bundle 346 is shown for illumination. The first fiber optic bundle 344 is shown having a circular shape. The second fiber optic bundle 346 is shown having an annular shape and is disposed about the first fiber optic bundle 344. These are just examples. Other shapes (e.g., circular, polygonal, rectangular, elliptical, etc.) and/or sizes are also contemplated. In some embodiments, the first fiber optic bundle 344 may align with and/or may be in communication with the imager disposed within the handle portion 310.

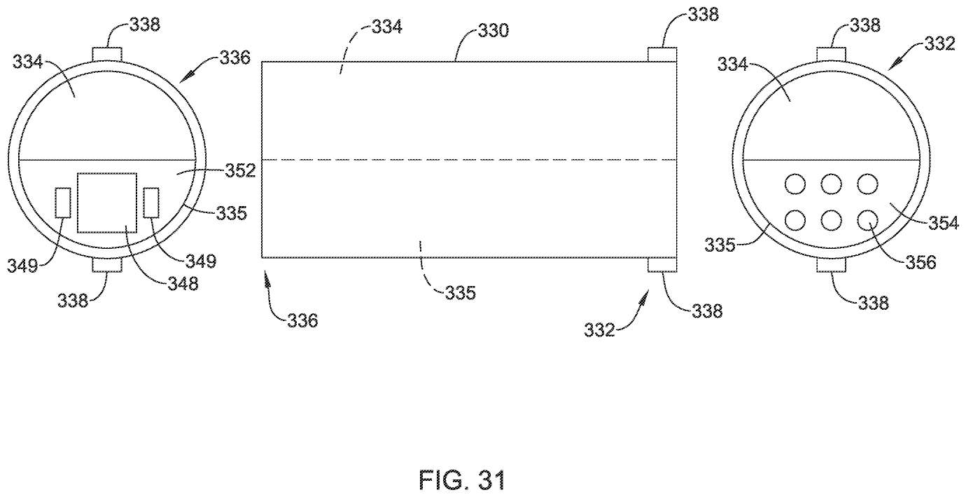

FIG. 31 illustrates distal end, side, and proximal end views of another example configuration for the disposable shaft portion 330. Some features described above and called out in the figure (e.g., the plurality of locking tabs 338, etc.) are not repeated in the interest of brevity. In some embodiments, the distal working channel 334 may be disposed within and/or extend along the top portion of the disposable shaft portion 330. In some embodiments, the elongate sleeve 335 may be disposed within and/or extend along a bottom portion of the disposable shaft portion 330. These are just examples. Other configurations are also contemplated.

In some embodiments, the disposable shaft portion 330 includes illumination and imaging components disposed at the distal end 336 of the disposable shaft portion 330. In the illustrated example, a CMOS imager 348 is disposed at the distal end 336 of the disposable shaft portion 330 and LEDs 349 for illumination are disposed at the distal end 336 of the disposable shaft portion 330. In some embodiments, the CMOS imager 348 and/or the LEDs 349 may be disposed on and/or may be fixedly attached to a distal printed circuit board 352 disposed at and/or within the distal end 336 of the disposable shaft portion 330. Further exemplary distal printed circuit boards are disclosed in U.S. Provisional Patent Application No. 63/083,151, the disclosure of which is incorporated herein by reference. The CMOS imager 348 is shown having a square shape. In some embodiments, the CMOS imager 348 may extend about 1 mm on each side. This is just an example. Other shapes (e.g., circular, polygonal, rectangular, elliptical, etc.) and/or sizes are also contemplated. In some embodiments, the distal printed circuit board 352 may be oriented perpendicular to a central longitudinal axis of the disposable shaft portion 330. In some embodiments, the distal printed circuit board 352 may be oriented at an oblique angle to the central longitudinal axis of the disposable shaft portion 330. For example, the distal printed circuit board 352 may be angled toward the distal working channel 334 of the disposable shaft portion 330.

In some embodiments, the disposable shaft portion 330 may include a proximal printed circuit board 354 disposed at and/or within the proximal end 332 of the disposable shaft portion 330. The proximal printed circuit board 354 may be in electronic and/or electrical communication with the distal printed circuit board 352. The proximal printed circuit board 354 may include a plurality of electrical pads 356 configured for electrical and/or electronic output from the disposable shaft portion 330 to the handle portion 310 and/or electrical and/or electronic input from the handle portion 310 to the disposable shaft portion 330. In at least some embodiments, the proximal printed circuit board 354 may align with and/or connect to a third printed circuit board disposed within the handle portion 310. In some embodiments, the handle portion 310 and/or the third printed circuit board may include spring loaded pins, e.g., pogo pins, configured and/or positioned to electrically connect with the plurality of electrical pads 356 on the proximal printed circuit board 354 of the disposable shaft portion 330.

It is contemplated that in some embodiments, the disposable shaft portion 330 may include the proximal printed circuit board 354 and not the distal printed circuit board 352. In some embodiments, the proximal printed circuit board 354 may be configured to control the illumination and imaging components disposed at the distal end of the disposable shaft portion 330, whether the illumination and imaging components are disposed on the distal printed circuit board 352 or at the distal end 336 of the disposable shaft portion 330 absent and/or without the distal printed circuit board 352. Other configurations are also contemplated.

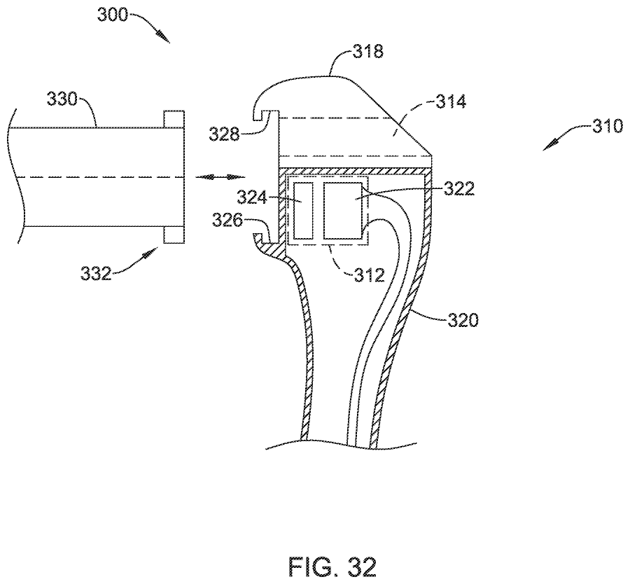

FIG. 32 is a partial cross-sectional view of an example configuration of the handle portion 310. In some embodiments, the handle portion 310 may include a reusable handle portion 320 and a disposable handle portion 318. The disposable handle portion 318 may be configured to releasably connect to the reusable handle portion 320. In one example, the disposable handle portion 318 may be configured to releasably connect to the reusable handle portion 320 via a twist-lock mechanism, a snap fit, a cam lock, etc. Other configurations are also contemplated. In some embodiments, the proximal working channel 314 may be disposed within the disposable handle portion 318. This may permit any portion of the modular medical device 300 that is expected to come into contact with tools used to treat the patient and/or with the patient's bodily fluids to be discarded after use (e.g., a single use device) instead of being subjected to cleaning and/or disinfection procedures. The more expensive elements of the modular medical device 300 may be disposed within the reusable handle portion 320 and thus cost per procedure may be reduced.

The electronic components 312 of the handle portion 310 may be disposed within the reusable handle portion 320. In some embodiments, the electronic components 312 may include a CMOS imager 322 and/or a lens 324. In some embodiments, the electronic components 312 may include LEDs and/or other elements. Other configurations are also contemplated, including various configurations that lack one or more of these features. In some embodiments, the disposable shaft portion 330 may be configured as shown in FIGS. 29-30. In some embodiments, the disposable shaft portion 330 may be configured as shown in FIG. 31. Other configurations are also contemplated, and the configuration and/or arrangement of elements in the handle portion 310 may be changed, modified, and/or adjusted according to the configuration of the disposable shaft portion 330 used in the modular medical device 300.

In some embodiments, when the proximal end 332 of the disposable shaft portion 330 is connected to the handle portion 310, the proximal end 332 of the disposable shaft portion 330 and/or the plurality of locking tabs 338 of the disposable shaft portion 330 may engage both the disposable handle portion 318 and the reusable handle portion 320. In some embodiments, the disposable handle portion 318 may be made from a lower cost material than the reusable handle portion 320. The reusable handle portion 320 and the disposable handle portion 318 may each be substantially rigid. In some embodiments, the reusable handle portion 320 may include one or more notches 326 configured to receive at least one of the plurality of locking tabs 338. In some embodiments, the disposable handle portion 318 may include one or more notches 328 configured to receive at least one of the plurality of locking tabs 338. In some embodiments, the one or more notches 326 in the reusable handle portion 320 and/or the one or more notches 328 in the disposable handle portion 318 may be spring-loaded or self-biased towards each other and/or towards the plurality of locking tabs 338 when the proximal end 332 of the disposable shaft portion 330 is connected to the handle portion 310. Other configurations are also contemplated.

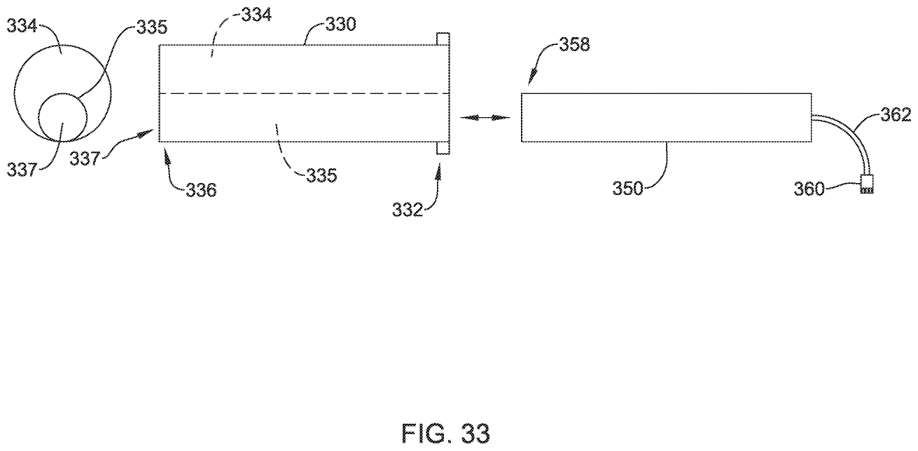

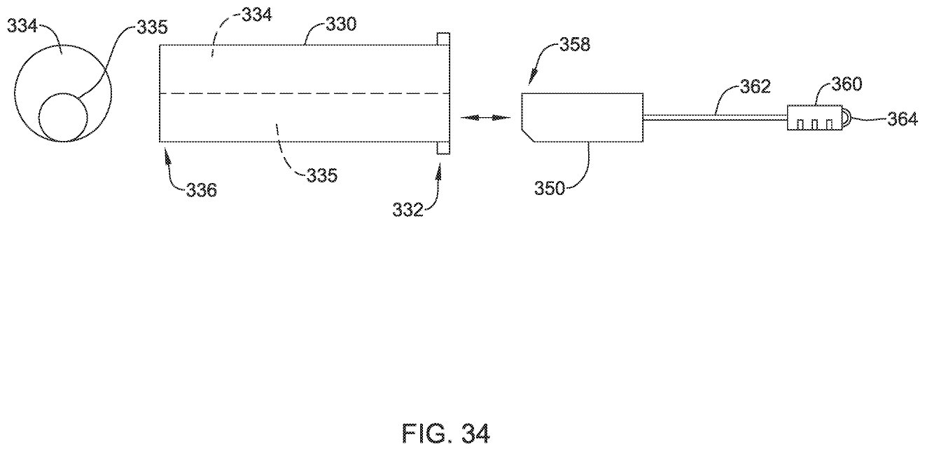

In some embodiments, the illumination and imaging components may be disposed on and/or within a reusable imaging assembly 350 removably and/or slidably disposed within the elongate sleeve 335 fixedly disposed within the disposable shaft portion 330, as shown in FIGS. 33-34. In an alternative configuration, the imaging assembly 350 may be disposed within a disposable polymeric sleeve configured to be inserted into the disposable shaft portion 330 and/or removed from the disposable shaft portion 330 along with and/or concurrently with the imaging assembly 350. The imaging assembly 350 may include a proximal connector 360 attached to the imaging assembly 350 by a cable 362. The proximal connector 360 of the imaging assembly 350 may be configured to connect to the electronic components 312 (not shown) in the handle portion 310 (not shown).

In some embodiments, a distal tip 358 of the imaging assembly 350 may include a CMOS imager, LEDs, and/or other elements. In some embodiments, the distal tip 358 may be constructed with an angled offset such that the CMOS imager does not face straight forward and/or distally. For example, the distal tip 358 may be constructed with a distal face oriented at an oblique angle to a central longitudinal axis of the imaging assembly 350. The angled offset may permit the practitioner to view a larger area adjacent the distal end 336 of the disposable shaft portion 330 if and/or when the imaging assembly 350 is rotated. In some embodiments, the imaging assembly 350 may be rotatably fixed relative to the disposable shaft portion 330 and/or the elongate sleeve 335. As such, the imaging assembly 350 may be rotated by rotating the modular medical device 300 (not shown) and/or the disposable shaft portion 330. In some embodiments, the imaging assembly 350 may be rotatable relative to the elongate sleeve 335 and/or the disposable shaft portion 330.

In some embodiments, the elongate sleeve 335 and/or the disposable polymeric sleeve may separate and/or protect the illumination and imaging components from the distal working channel 334 and/or the patient's bodily fluids and/or fluids or materials used during the medical procedure. Accordingly, in some embodiments, the elongate sleeve 335 may include a transparent window 337 disposed across and/or fixedly attached to a distal end of the elongate sleeve 335, as seen in FIG. 33. In some embodiments, the transparent window 337 may be disposed at the distal end 336 of the disposable shaft portion 330. In some embodiments, the transparent window 337 may be formed from a polymeric material, glass, or another suitable transparent and biocompatible material. Similarly, the disposable polymeric sleeve may include a transparent distal end configured to cooperate with the illumination and imaging components. In some embodiments, the elongate sleeve 335 and/or the transparent window 337 may be impervious to fluids and/or may be sealed off from the distal working channel 334 and/or may prevent the patient's bodily fluids and/or fluids or materials used during the medical procedure from entering the elongate sleeve 335 and/or contacting the imaging assembly 350. In some embodiments, the transparent window 337 may include an anti-reflective coating disposed on an inside surface of the transparent window 337 to improve visualization through the transparent window 337.

In some embodiments, since the reusable imaging assembly 350 is not intended to contact the patient's bodily fluids and/or any tools used to treat the patient, the illumination and imaging components disposed on and/or within the imaging assembly 350 may be configured to be reusable via a simple wipe down and/or other appropriate disinfection procedures, while the disposable shaft portion 330 may be discarded after use. By saving and reusing the relatively more expensive illumination and imaging components, cost per use and/or procedure may be reduced.

In at least some embodiments, where the elongate sleeve 335 and/or the transparent window 337 are used for example, the reusable imaging assembly 350 may be configured to be inserted and/or pushed into the elongate sleeve 335 at the proximal end 332 of the disposable shaft portion 330, as seen in FIG. 33. The imaging assembly 350 may be configured to slide distally toward the distal end 336 of the disposable shaft portion 330 within the elongate sleeve 335. In some embodiments, the imaging assembly 350 may have a body length that is substantially similar to and/or equal to a length of the elongate sleeve 335 and/or the disposable shaft portion 330. The imaging assembly 350 may be semi-rigid and/or rigid along its body length to facilitate inserting and/or pushing the imaging assembly 350 into position within the elongate sleeve 335 and/or the disposable shaft portion 330.

In some embodiments, a distal end of the imaging assembly 350 may be positioned at, adjacent to, and/or against the transparent window 337. In some embodiments, the transparent window 337 may prevent over insertion of the imaging assembly 350. In some embodiments, the imaging assembly 350 may include a proximal flange extending radially outward and configured to engage the proximal end 332 of the disposable shaft portion 330 and/or a proximal end of the elongate sleeve 335 when the distal tip 358 of the imaging assembly 350 is disposed at, adjacent to, and/or against the transparent window 337.

In some embodiments, the imaging assembly 350 may be configured to be pulled through the elongate sleeve 335 from the distal end 336 of the disposable shaft portion 330, as shown in FIG. 34. Accordingly, in a pull-through configuration, the elongate sleeve 335 and/or the disposable shaft portion 330 may be devoid of the transparent window 337. As such, in the pull-through configuration, the disposable sleeve may be used to protect the imaging assembly 350 from contact with the patient's bodily fluids and/or tools used to treat the patient. Alternatively, the imaging assembly 350 may be configured to be thoroughly cleaned and sterilized after use according to accepted procedures.

In the pull-through configuration, the imaging assembly 350 may have a body length that is shorter than the elongate sleeve 335 and/or the disposable shaft portion 330. The imaging assembly 350 does not necessarily need to be semi-rigid and/or rigid along its body length because the imaging assembly 350 is pulled into position within the elongate sleeve 335 and/or the disposable shaft portion 330 instead of pushed. To facilitate pulling the imaging assembly 350 through the elongate sleeve 335 and/or the disposable shaft portion 330, the proximal connector 360 may include a hook 364 fixedly attached thereto, as seen in FIG. 34. A pull wire or other device may be engaged with the hook 364 and thereafter used to pull the imaging assembly 350 into position within the elongate sleeve 335 and/or the disposable shaft portion 330.

In some alternative embodiments, the imaging assembly 350 may be configured to screw into position within the elongate sleeve 335 and/or the disposable shaft portion 330 via helical threads. In other alternative embodiments, the imaging assembly 350 may be configured to twist into position with locking tabs and corresponding notches. Other configurations are also contemplated.

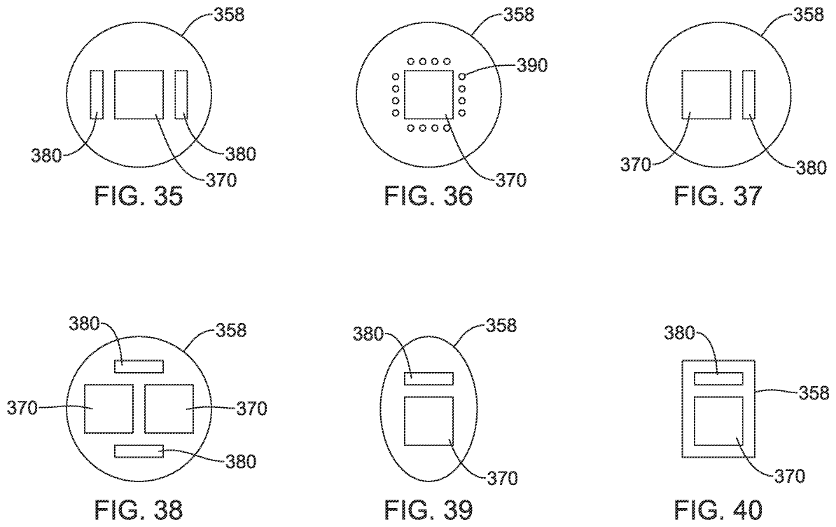

FIGS. 35-40 illustrates various configurations for the illumination and imaging components disposed at and/or within the distal tip 358 of the imaging assembly 350. Additionally, one or more of these configurations may be used in the disposable shaft portion 330 illustrated in FIGS. 29-31. In any one configuration, the distal tip 358 may include a CMOS imager 370, one or more LED 380, and/or a plurality of optical fibers and/or fiber optic bundles 390. In some embodiments, the CMOS imager 370 may be generally square in shape and may extend about 0.65 mm on each side. This is just an example. Other shapes, sizes, and/or configurations are also contemplated. In some embodiments, the one or more LED 380 may be generally rectangular in shape and may extend about 0.35 mm on its short side(s) and about 0.65 mm on its long side(s). This is just an example. Other shapes, sizes, and/or configurations are also contemplated. Furthermore, in some embodiments, components supporting stereo imaging, specified wavelength imaging, ultrasound, and/or other sensors may be provided in addition to or in place of other components.

FIG. 35 illustrates the distal tip 358 having a substantially circular cross-section. In some embodiments, the circular cross-section may have an outer diameter of about 2.2 mm. This is just an example. Other sizes and/or configurations are also contemplated. In some embodiments, the distal tip 358 may include a first CMOS imager 370 disposed along a central longitudinal axis of the imaging assembly 350. In some embodiments, the first CMOS imager 370 may be centered on the central longitudinal axis of the imaging assembly 350. The distal tip 358 may include a first LED 380 and a second LED 380. The first LED 380 and the second LED 380 may be disposed on opposite sides of the first CMOS imager 370. In some embodiments, the first LED 380 and the second LED 380 may be spaced apart from the first CMOS imager 370. In some embodiments, the first LED 380 and the second LED 380 may be spaced equidistantly from the central longitudinal axis of the imaging assembly 350. In some embodiments, the first LED 380 and the second LED 380 may be disposed immediately adjacent to and/or in physical contact with the first CMOS imager 370. In some embodiments, a major axis of the first LED 380 may be oriented parallel to a side of the first CMOS imager 370 adjacent to and/or closest to the first LED 380, and a major axis of the second LED 380 may be oriented parallel to a side of the first CMOS imager 370 adjacent to and/or closest to the second LED 380. Other configurations are also contemplated.

FIG. 36 illustrates the distal tip 358 having a substantially circular cross-section. In some embodiments, the circular cross-section may have an outer diameter of about 1.2 mm. This is just an example. Other sizes and/or configurations are also contemplated. In some embodiments, the distal tip 358 may include a first CMOS imager 370 disposed along a central longitudinal axis of the imaging assembly 350. In some embodiments, the first CMOS imager 370 may be centered on the central longitudinal axis of the imaging assembly 350. The distal tip 358 may include a plurality of optical fibers and/or fiber optic bundles 390. The plurality of optical fibers and/or fiber optic bundles 390 may be disposed along and/or around the sides of the first CMOS imager 370. In some embodiments, the plurality of optical fibers and/or fiber optic bundles 390 may be spaced apart from the first CMOS imager 370 and each other. In some embodiments, the plurality of optical fibers and/or fiber optic bundles 390 may be disposed in straight lines parallel to the sides of the first CMOS imager 370. In some embodiments, the plurality of optical fibers and/or fiber optic bundles 390 may be spaced equidistantly from the sides of the first CMOS imager 370 and from each other along each side. In some embodiments, the plurality of optical fibers and/or fiber optic bundles 390 may be disposed immediately adjacent to and/or in physical contact with the first CMOS imager 370. In some embodiments, the plurality of optical fibers and/or fiber optic bundles 390 may be arranged in a circle around the first CMOS imager 370. In some embodiments, the plurality of optical fibers and/or fiber optic bundles 390 may be spaced equidistantly from the central longitudinal axis of the imaging assembly 350. Other configurations are also contemplated.

FIG. 37 illustrates the distal tip 358 having a substantially circular cross-section. In some embodiments, the circular cross-section may have an outer diameter of about 1.7 mm. This is just an example. Other sizes and/or configurations are also contemplated. In some embodiments, the distal tip 358 may include a first CMOS imager 370 disposed along a central longitudinal axis of the imaging assembly 350. In some embodiments, a center of the first CMOS imager 370 may be offset laterally from the central longitudinal axis of the imaging assembly 350 in a first direction. The distal tip 358 may include a first LED 380 disposed along a side of the first CMOS imager 370 closest to the central longitudinal axis of the imaging assembly 350. In some embodiments, a center of the first LED 380 may be laterally offset from the central longitudinal axis of the imaging assembly 350 in a second direction opposite the first direction. In some embodiments, the first LED 380 may be spaced apart from the first CMOS imager 370. In some embodiments, the first LED 380 may be disposed immediately adjacent to and/or in physical contact with the first CMOS imager 370. In some embodiments, a major axis of the first LED 380 may be oriented parallel to a side of the first CMOS imager 370 adjacent to and/or closest to the first LED 380. Other configurations are also contemplated.

FIG. 38 illustrates the distal tip 358 having a substantially circular cross-section. In some embodiments, the circular cross-section may have an outer diameter of about 2.2 mm. This is just an example. Other sizes and/or configurations are also contemplated. In some embodiments, the distal tip 358 may include a first CMOS imager 370 and a second CMOS imager 370 each disposed along a central longitudinal axis of the imaging assembly 350. In some embodiments, a center of the first CMOS imager 370 may be laterally offset from the central longitudinal axis of the imaging assembly 350 in a first direction, and a center of the second CMOS imager 370 may be laterally offset from the central longitudinal axis of the imaging assembly 350 in a second direction opposite the first direction. In at least some embodiments, the first CMOS imager 370 and the second CMOS imager 370 may collectively form a stereo imager. The distal tip 358 may include a first LED 380 and a second LED 380. The first LED 380 and the second LED 380 may be disposed on opposite sides of an axis connecting the center of the first CMOS imager 370 and the center of the second CMOS imager 370. In some embodiments, the first LED 380 and the second LED 380 may be spaced apart from the first CMOS imager 370 and/or the second CMOS imager 370. In some embodiments, the first LED 380 and the second LED 380 may be spaced equidistantly from the central longitudinal axis of the imaging assembly 350. In some embodiments, the first LED 380 and the second LED 380 may be disposed immediately adjacent to and/or in physical contact with the first CMOS imager 370 and/or the second CMOS imager 370. In some embodiments, a major axis of the first LED 380 may be oriented parallel to the axis connecting the center of the first CMOS imager 370 and the center of the second CMOS imager 370, and a major axis of the second LED 380 may be oriented parallel to the axis connecting the center of the first CMOS imager 370 and the center of the second CMOS imager 370. Other configurations are also contemplated.

FIG. 39 illustrates the distal tip 358 having a substantially elliptical, oval, and/or oblong cross-section. In some embodiments, the elliptical, oval, and/or oblong cross-section may have a major diameter of about 1.6 mm and a minor diameter of about 1.3 mm. This is just an example. Other sizes and/or configurations are also contemplated. In some embodiments, the distal tip 358 may include a first CMOS imager 370 disposed along a central longitudinal axis of the imaging assembly 350. In some embodiments, a center of the first CMOS imager 370 may be offset laterally from the central longitudinal axis of the imaging assembly 350 in a first direction. The distal tip 358 may include a first LED 380 disposed along a side of the first CMOS imager 370 closest to the central longitudinal axis of the imaging assembly 350. In some embodiments, a center of the first LED 380 may be laterally offset from the central longitudinal axis of the imaging assembly 350 in a second direction opposite the first direction. In some embodiments, the first LED 380 may be spaced apart from the first CMOS imager 370. In some embodiments, the first LED 380 may be disposed immediately adjacent to and/or in physical contact with the first CMOS imager 370. In some embodiments, a major axis of the first LED 380 may be oriented parallel to a side of the first CMOS imager 370 adjacent to and/or closest to the first LED 380. Other configurations are also contemplated.

FIG. 40 illustrates the distal tip 358 having a substantially rectangular cross-section. In some embodiments, the rectangular cross-section may extend about 1.3 mm along its long side(s) and may extend about 0.9 mm along its short side(s). This is just an example. Other sizes and/or configurations are also contemplated. In some embodiments, the distal tip 358 may include a first CMOS imager 370 disposed along a central longitudinal axis of the imaging assembly 350. In some embodiments, a center of the first CMOS imager 370 may be offset laterally from the central longitudinal axis of the imaging assembly 350 in a first direction. The distal tip 358 may include a first LED 380 disposed along a side of the first CMOS imager 370 closest to the central longitudinal axis of the imaging assembly 350. In some embodiments, a center of the first LED 380 may be laterally offset from the central longitudinal axis of the imaging assembly 350 in a second direction opposite the first direction. In some embodiments, the first LED 380 may be spaced apart from the first CMOS imager 370. In some embodiments, the first LED 380 may be disposed immediately adjacent to and/or in physical contact with the first CMOS imager 370. In some embodiments, a major axis of the first LED 380 may be oriented parallel to a side of the first CMOS imager 370 adjacent to and/or closest to the first LED 380. Other configurations are also contemplated.

The materials that can be used for the various components of the medical systems and the various elements thereof disclosed herein may include those commonly associated with medical system and/or devices. For simplicity purposes, the following discussion refers to the medical system. However, this is not intended to limit the devices and methods described herein, as the discussion may be applied to other elements, members, components, or devices disclosed herein, such as but not limited to the access sheath 100, the obturator 120, the bridge 130, the access sheath 150, the guidewire 158, the flexible ureteroscope 160, the stent 190, the elongate adapter element 200, the attachment portion 270, the modular medical device 300, the handle portion 310, disposable shaft portion 330, the imaging assembly 350, and/or elements or components thereof.

In some embodiments, the medical system and/or components thereof may be made from a metal, metal alloy, polymer (some examples of which are disclosed below), a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyurethane silicone copolymers (for example, ElastEon® from AorTech Biomaterials or ChronoSil® from AdvanSource Biomaterials), biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In some embodiments, a linear-elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Other suitable materials may include ULTANIUM™ (available from Neo-Metrics) and GUM METAL™ (available from Toyota). In some other embodiments, a super-elastic alloy, for example a superelastic nitinol can be used to achieve desired properties.

In at least some embodiments, portions or all of the medical system and/or components thereof, may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the medical system in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the medical system to achieve the same result.

In some embodiments, a degree of Magnetic Resonance Imaging (Mill) compatibility is imparted into the medical system and/or other elements disclosed herein. For example, the medical system and/or components or portions thereof may be made of a material that does not substantially distort the image and create substantial artifacts (i.e., gaps in the image). Certain ferromagnetic materials, for example, may not be suitable because they may create artifacts in an MRI image. The medical system or portions thereof may also be made from a material that the Mill machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

In some embodiments, the medical system and/or other elements disclosed herein may include a fabric material disposed over or within the structure. The fabric material may be composed of a biocompatible material, such a polymeric material or biomaterial, adapted to promote tissue ingrowth. In some embodiments, the fabric material may include a bioabsorbable material. Some examples of suitable fabric materials include, but are not limited to, polyethylene glycol (PEG), nylon, polytetrafluoroethylene (PTFE, ePTFE), a polyolefinic material such as a polyethylene, a polypropylene, polyester, polyurethane, and/or blends or combinations thereof.

In some embodiments, the medical system and/or other elements disclosed herein may include and/or be formed from a textile material. Some examples of suitable textile materials may include synthetic yarns that may be flat, shaped, twisted, textured, pre-shrunk or un-shrunk. Synthetic biocompatible yarns suitable for use in the present disclosure include, but are not limited to, polyesters, including polyethylene terephthalate (PET) polyesters, polypropylenes, polyethylenes, polyurethanes, polyolefins, polyvinyls, poly methyl acetates, polyamides, naphthalene dicarboxylene derivatives, natural silk, and polytetrafluoroethylenes. Moreover, at least one of the synthetic yarns may be a metallic yarn or a glass or ceramic yarn or fiber. Useful metallic yarns include those yarns made from or containing stainless steel, platinum, gold, titanium, tantalum or a Ni—Co—Cr-based alloy. The yarns may further include carbon, glass or ceramic fibers. Desirably, the yarns are made from thermoplastic materials including, but not limited to, polyesters, polypropylenes, polyethylenes, polyurethanes, polynaphthalenes, polytetrafluoroethylenes, and the like. The yarns may be of the multifilament, monofilament, or spun types. The type and denier of the yarn chosen may be selected in a manner which forms a biocompatible and implantable prosthesis and, more particularly, a vascular structure having desirable properties.

In some embodiments, the medical system and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethylketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); cholesterol-lowering agents;

vasodilating agents; and agents which interfere with endogenous vasoactive mechanisms.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. A method of treating a kidney of a patient, comprising:
advancing an access sheath through a urethra into a bladder of the patient;
disposing a flexible ureteroscope within a lumen of the access sheath;
visually locating a ureteric orifice within the bladder using the flexible ureteroscope while the flexible ureteroscope is disposed within the lumen of the access sheath;
inserting a guidewire into a working lumen the flexible ureteroscope;
advancing the guidewire from the working lumen into the ureteric orifice while the flexible ureteroscope is disposed within the lumen of the access sheath;
advancing the flexible ureteroscope through the ureteric orifice and into the kidney while the flexible ureteroscope is disposed within the lumen of the access sheath; and
performing a procedure within the kidney using the flexible ureteroscope while the flexible ureteroscope is disposed within the lumen of the access sheath.

2. The method of claim 1, further comprising:
removing the flexible ureteroscope from the lumen of the access sheath while maintaining a distal end of the guidewire within the kidney; and
advancing a deflectable distal tip of the flexible ureteroscope through the lumen of the access sheath alongside the guidewire and into the bladder.

3. The method of claim 2, further comprising:
advancing a stent over the guidewire while the flexible ureteroscope is disposed within the lumen of the access sheath; and
positioning a distal end of the stent within the kidney while the flexible ureteroscope is disposed within the lumen of the access sheath.

4. The method of claim 3, further comprising:
visually confirming placement of the stent using the flexible ureteroscope while the flexible ureteroscope is disposed within the lumen of the access sheath.

5. The method of claim 1, wherein disposing a flexible ureteroscope within a lumen of the access sheath includes aligning at least one crosshair marking on an elongate shaft of the flexible ureteroscope with a corresponding crosshair marking on the access sheath and locking an orientation of the flexible ureteroscope relative to the access sheath.

6. The method of claim 1, further comprising:
disposing an access sheath over the guidewire; and
advancing the access sheath into the ureteric orifice while the flexible ureteroscope is disposed within the lumen of the access sheath.

7. The method of claim 6, wherein disposing an access sheath over the guidewire includes disposing the access sheath over an elongate shaft of the flexible ureteroscope; and
wherein advancing the access sheath into the ureteric orifice while the flexible ureteroscope is disposed within the lumen of the access sheath includes advancing the access sheath over the elongate shaft of the flexible ureteroscope into the ureteric orifice after advancing the flexible ureteroscope through the ureteric orifice and into the kidney while the flexible ureteroscope is disposed within the lumen of the access sheath.

8. A medical system for treating a urinary tract of a patient, comprising:
a flexible ureteroscope including an elongate shaft having a deflectable distal tip, a working lumen extending therethrough, and optics disposed in the deflectable distal tip and in electronic communication with a handle of the flexible ureteroscope; and
an elongate adapter element configured to removably attach to the elongate shaft;
wherein the elongate adapter element includes a working channel extending from a proximal end of the elongate adapter element to a distal end of the elongate adapter element and an open slot extending along a majority of a length of the elongate adapter element;
wherein the elongate adapter element is configured to apply a radially inward force on an outer surface of the elongate shaft when at least a portion of the elongate shaft is disposed within the open slot;
wherein the working channel is circumferentially enclosed from the proximal end of the elongate adapter element to the distal end of the elongate adapter element and has a first cross-sectional area;
wherein the open slot has a second cross-sectional area greater than the first cross-sectional area.

9. The medical system of claim 8, wherein when at least a portion of the elongate shaft is disposed within the open slot, the elongate adapter element extends circumferentially around at least 60% of a circumference of the elongate shaft.

10. The medical system of claim 8, wherein when at least a portion of the elongate shaft is disposed within the open slot, the elongate adapter element extends circumferentially around at least 75% of a circumference of the elongate shaft.

11. The medical system of claim 8, wherein the elongate adapter element is configured to be positioned proximal of the deflectable distal tip when at least a portion of the elongate shaft is disposed within the open slot.

12. The medical system of claim 8, wherein the elongate adapter element includes a support member configured to secure excess length of the elongate shaft relative to the elongate adapter element.

13. The medical system of claim 12, wherein the support member forms the excess length of the elongate shaft into a loop.

14. The medical system of claim 8, wherein the elongate adapter element includes an attachment portion configured to removably attach to the handle of the flexible ureteroscope.

15. The medical system of claim 8, wherein the elongate adapter element includes a laterally facing cutout in communication with the open slot and disposed between the proximal end of the elongate adapter element and a longitudinal midpoint of the elongate adapter element.

16. The medical system of claim 15, wherein when at least a portion of the elongate shaft is disposed within the open slot, a portion of the elongate shaft extends through the laterally facing cutout and is positioned outside of the elongate adapter element.

17. A modular medical device, comprising:
a handle portion; and
a disposable shaft portion configured to releasably connect to the handle portion;
wherein the handle portion includes electronic components configured to communicate with the disposable shaft portion when the disposable shaft portion is connected to the handle portion;
wherein the handle portion includes a proximal working channel extending through the handle portion;
wherein the disposable shaft portion includes a distal working channel configured to be in fluid communication with the proximal working channel when the disposable shaft portion is connected to the handle portion;
wherein the disposable shaft portion includes illumination and imaging components disposed at a distal end of the disposable shaft portion;
wherein the illumination and imaging components are in electronic communication with the electronic components when the disposable shaft portion is connected to the handle portion;
wherein the illumination and imaging components are configured to illuminate and image an area proximate the distal end of the disposable shaft portion.

18. The modular medical device of claim 17, wherein the disposable shaft portion includes a printed circuit board disposed at the proximal end thereof, wherein the printed circuit board is configured to control the illumination and imaging components.

19. The modular medical device of claim 17, wherein the handle portion includes a reusable handle portion and a disposable handle portion;
wherein the proximal working channel is disposed within the disposable handle portion;
wherein the electronic components are disposed within the reusable handle portion;
wherein when the disposable shaft portion is connected to the handle portion, the proximal end of the disposable shaft portion engages both the disposable handle portion and the reusable handle portion.

20. The modular medical device of claim 17, wherein the illumination and imaging components are disposed on an imaging assembly removably disposed within an elongate sleeve fixedly disposed within the disposable shaft portion.
